(19) United States
(12) Patent Application Publication
(10) Pub. No.: US 2014/0051701 A1
(43) Pub. Date: Feb. 20, 2014

(54) METHODS OF TREATING A DISEASE OR CONDITION OF THE CENTRAL NERVOUS SYSTEM

(71) Applicants: Sue O'Connor, Prospect (AU); Andrew John Harvey, Goodwood (AU); Stephanie Wagner, Barr (FR); Emile Andriambeloson, Strasbourg (FR)

(72) Inventors: Sue O'Connor, Prospect (AU); Andrew John Harvey, Goodwood (AU); Stephanie Wagner, Barr (FR); Emile Andriambeloson, Strasbourg (FR)

(73) Assignee: Bionomics Limited, Thebarton (AU)

(21) Appl. No.: 14/002,536

(22) PCT Filed: Mar. 2, 2012
(86) PCT No.: PCT/AU2012/000216

§ 371 Date: Nov. 5, 2013

(30) Foreign Application Priority Data
Mar. 2, 2011 (AU) 2011900737

(51) Int. Cl. C07D 471/04

(52) U.S. Cl. CPC C07D 471/04
USPC 514/234.5; 514/253.04; 514/300

(57) ABSTRACT

The present invention provides methods of treating central nervous system disorders, such as mood disorders (e.g., depression) and neurodegenerative diseases using compounds of formula (I). The subject disclosure enables the manufacture of medicaments as well as compositions containing same for use in methods of therapy and prophylaxis of central nervous system disorders.

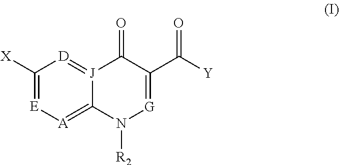

### RELATED APPLICATION

The present application claims priority to Australian provisional application number 2011900737, filed Mar. 2, 2011, which is incorporated herein by reference in its entirety.

### FIELD

The present disclosure relates generally to the treatment of central nervous system disorders, such as mood disorders (e.g., depression) and neurodegenerative diseases. The subject disclosure enables the manufacture of medicaments as well as compositions containing same for use in methods of therapy and prophylaxis of central nervous system disorders.

### BACKGROUND OF THE INVENTION

A neurite is any projection or outgrowth emanating from the cell body of a neuron or nerve cell. Neurons are the core components of the nervous system, which includes the brain, spinal cord, and peripheral ganglia. Compounds inducing neurite outgrowth have neuroprotective properties and the induction of neurite outgrowth is a surrogate of the ability of a compound to induce neurogenesis.

Neurotrophins are critical mediators of neuronal survival during development and are involved in the regulation of neurogenesis (axonal and dendritic outgrowth), synapse formation and function, cell migration and cell proliferation, plasticity, survival and differentiation in adult neurons and glia. Although the majority of neurons in the mammalian brain are formed prenatally, parts of the adult brain retain the ability to grow new neurons from neural stem cells in a process known as neurogenesis.

Neurotrophins are highly specific ligands for Trk (tropomyosin receptor-kinase) receptors, the most common of which are TrkA, TrkB, and TrkC. Each type of neurotrophin has a different binding affinity toward its corresponding Trk receptor. TrkA is a signaling receptor for nerve growth factor (NGF), TrkB is a signaling receptor for the related neurotrophin brain-derived neurotrophic factor (BDNF), neurotrophin 4/5 and, with lower affinity, for neurotrophin-3, and TrkC is a receptor for neurotrophin-3 (NT3). The activation of Trk receptors by the binding of specific neurotrophins triggers receptor dimerization and consequent trans-phosphorylation of tyrosine residues of the tyrosine kinase domain. Phosphorylated receptors undergo conformational changes which promote the recruitment of intracellular substrates such SHC1, PI-3 kinase and PLCγ-1 to activate signaling cascades. For example, the recruitment and tyrosine phosphorylation of PLCγ-1 activates this enzyme and catalyzes the breakdown of lipids to diacyl glycerol and inositol(1,4,5)triphosphate (IP3). Binding of IP3 to specific receptors promotes release of calcium from intracellular stores, while diacyl glycerol allows maximal activation of several protein kinase C isoforms. In addition, the phospholipase pathway can indirectly activate MAP kinases and phosphatidylinositol 30-kinase (PI3 kinase) by changes in intracellular calcium. These intracellular signal cascades may result in neurogenesis, promotion of neuronal survival during development and following injuries, neuronal differentiation and maintenance, control short-term and long-term synaptic activity and other functional regulation of cells.

Antidepressants (e.g., SSRIs and tricyclics) and mood stabilisers (sodium valproate, lithium) have been found to exhibit neurotrophic properties. These effects are not directly mediated through Trk receptors but occur via activation of neurotrophic signalling pathways that trigger biological events within the cell to modulate neuronal function. Activation of G-protein coupled receptors, for example, initiates signalling from many downstream effector proteins, such as phospholipases and ion channels, thus permitting the release of second messenger molecules within the cell, such as IP3 or calcium ions to promote neurogenesis.

Neurodegenerative diseases are characterised by a loss of neurons from specific regions of the central nervous system. Current research has provided evidence that neurogenesis is impaired in neurodegenerative diseases such as Parkinson's disease, Lewy body disease, and Huntington's disease, and amyotrophic lateral sclerosis, and that stimulation of neurogenesis is associated with restored function in animal models of these diseases, suggesting that neurogenesis is functionally important.

From the above it can be observed that neurite outgrowth is a critical event in neuronal development, the formation and remodelling of synapses, response to injury, and regeneration. Changes in the pattern of neurite outgrowth have been implicated in neurodegenerative disorders including traumatic brain injury. The discovery of new compounds that can positively affect neurite outgrowth by directly modulating neurotrophic pathways is important for the development of new therapeutic agents for treating certain central nervous system disorders (including mood disorders, such as depression, neurodegenerative diseases, and brain injury).

### SUMMARY

The instant disclosure teaches that compounds of formula (I) act as effective enhancers of neurite outgrowth in animals including mammals (such as human) and are therefore therapeutically useful in the prophylaxis and treatment of certain central nervous system (CNS) disorders, such as mood disorders (e.g., depression) and neurodegenerative diseases.

By “disorder” includes an adverse condition, trauma or other adverse manifestation of the CNS.

Accordingly, provided herein is a method of enhancing neurite outgrowth in a subject in need thereof, the method including the step of administering an effective amount of a compound of formula (I) or pharmaceutically acceptable salt thereof:

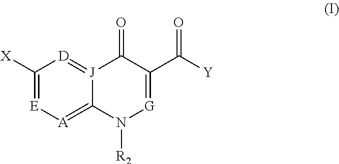

where A, E, G and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N;
   J represents C or N;
   X represents halogen, optionally substituted heteroaryl, OR₁, or NR₁R″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heteroaryl, optionally substituted oxysulfinyl, optionally substituted oxysulfonyl, optionally substituted sulfinyl, and optionally substituted sulfonyl);
   Y represents OR′″ (where R′″ is H or optionally substituted alkyl) or NR₃R₄;
   R₁ represents H, optionally substituted cycloalkyl, optionally substituted cycloalkenyl, optionally substituted alkyl, optionally substituted acyl, optionally substituted aryl, optionally substituted heterocyclyl, or optionally substituted heteroaryl;
   R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
   R₃ and R₄ each independently represent H, optionally substituted alkyl, optionally substituted cycloalkyl, optionally substituted aryl, optionally substituted heteroaryl, or optionally substituted heterocyclyl, or together with the N-atom optionally substituted N-containing heteroaryl or optionally substituted N-containing heterocyclyl.
   Also provided is a method for the treatment or prophylaxis of depression in a subject in need thereof, the method including the step of administering to said subject a compound of formula (I) or a pharmaceutically acceptable salt thereof;

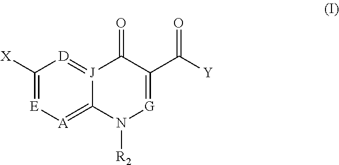

where A, E, G, D, J, X, Y and R₂ are as described herein.

In certain embodiments, the depression is a symptom of a neurodegenerative disease.

Also provided is a method for the treatment or prophylaxis of a neurodegenerative disease in a subject in need thereof, the method including the step of administering to said subject a compound of formula (I) or a pharmaceutically acceptable salt thereof;

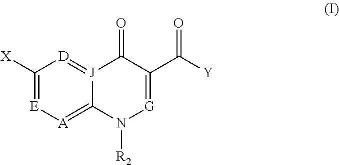

where A, E, G, D, J, X, Y and R₂ are as described herein.

The compounds of formula (I) or pharmaceutically acceptable salts thereof are generally given for a time and under conditions sufficient to treat the disease, prevent or delay onset or development of the disease, or treat or prevent symptoms of the disease.

Also provided herein is a method of treating or preventing relapse of depression in a subject receiving antidepressant therapy, or in a subject having a history of depression, the method including the step of administering to said subject a compound of formula (I) or a pharmaceutically acceptable salt thereof.

Also provided herein is a method of treating or preventing depression in a subject in need thereof, the method including the step of administering to said subject a compound of formula (I), or a pharmaceutically acceptable salt thereof, in the absence of adjunct antidepressant therapy.

The present disclosure further enables the use of a compound of formula (I), or a pharmaceutically acceptable salt thereof, in the manufacture of a medicament for enhancing neurite outgrowth in a subject.

The present disclosure further enables the use of a compound of formula (I), or a pharmaceutically acceptable salt thereof, in the manufacture of a medicament for the treatment or prophylaxis of depression in a subject in need thereof.

Also provided herein is the use of a compound of formula (I), or a pharmaceutically acceptable salt thereof, in the manufacture of a medicament for treating or preventing relapse of depression in a subject receiving antidepressant therapy.

Also provided herein is the use of a compound of formula (I), or a pharmaceutically acceptable salt thereof, in the manufacture of a medicament for treating, preventing, or delaying onset or development of a disease of the CNS in a subject.

Also provided herein is the use of a compound of formula (I), or a pharmaceutically acceptable salt thereof, in the manufacture of a medicament for treating or preventing depression in a subject in need thereof, the method including the step of administering to said subject a compound of formula (I), or a pharmaceutically acceptable salt thereof, in the absence of adjunct antidepressant therapy.

The present disclosure further teaches the use of a compound of formula (I), or a pharmaceutically acceptable salt thereof, in the manufacture of a medicament for treating or preventing or delaying onset or development of a neurodegenerative disease in a subject.

Also provided is the use of a compound of formula (I), or a pharmaceutically acceptable salt thereof, in the manufacture of a medicament for ameliorating the symptoms of a disease of the CNS, such as a neurodegenerative disease.

Also provided is the use of a compound of formula (I), or a pharmaceutically acceptable salt thereof, in the manufacture of a medicament for ameliorating the symptoms of a disease of the CNS, such as depression. In certain embodiments, the depression is a symptom of a neurodegenerative disease.

Other symptoms of a disease of the CNS include, but are not limited to, cognitive impairment (e.g., memory loss), headaches, sensory loss, motor dysfunction, tremors, seizures and slurred speech.

### BRIEF DESCRIPTION OF THE FIGURES

FIG. 1 shows the effect of the compound of Example 1 (Compound 1) on neurite outgrowth in vitro. The compound of Example 1 was tested at 0.1 nM, 1 nM, 10 nM, 100 nM and 1000 nM on neurite cell cultures comprising two Petri dishes per culture and per condition. In parallel, brain-derived neurotrophic factor (BDNF) was tested at 50 ng/ml. *p≦0.05, significantly different compared to control.

FIG. 2 shows the effect of the compounds of Example 2 (Compound 2) (FIG. 2B) and Example 3 (Compound 3) (FIG. 2C) on neurite outgrowth in vitro, as compared to control and Compound 1 (FIG. 2A). *p≦0.05, significantly different compared to control.

FIG. 3 shows that enhancement of neurite outgrowth by Compound 1 in rat primary cortical neurons is blocked by the PLC inhibitor, U73122. FIG. 3A—from left to right: Control; Compound 1 (10 nM); U73122 (1 μM); U73122 (1 μM)+Compound 1 (10 nM). ***p≦0.0001, significantly different compared to the Control group. FIG. 3B—from left to right: Control; Compound 1 (10 nM); U73122 (0.03 μM); U73122 (0.1 μM); U73122 (0.3 μM); U73122 (1 μM); Compound 1 (10 nM)+U73122 (0.03 μM); Compound 1 (10 nM)+U73122 (0.1 μM); Compound 1 (10 nM)+U73122 (0.3 μM); Compound 1 (10 nM)+U73122 (1 μM). *p≦0.05 and ***p≦0.0001, significantly different compared to the control group. ̂̂̂p≦0.0001, significantly different compared to Compound 1 treatment alone; n=150-180 cells.

FIG. 4 shows that U73122 blocks Compound 1 activity in the mouse light dark box model. For each of FIGS. 4A-4C—from left to right: Vehicle; U73122 (30 mg/kg i.p.); Compound 1 (10 mg/kg, p.o.); U73122+Compound 1. **p≦0.01, p*** p≦0.0001 significantly different compared to control group, ̂̂p≦0.01 ̂̂̂p≦0.0001 significantly different compared to Compound 1 treatment alone (N=10 mice).

FIG. 5 shows that U73122 blocks Compound 1, but not diazepam, activity in the mouse light dark box model. For each of FIGS. 5A-5C—from left to right: Vehicle; U73122 (30 mg/kg i.p.); Compound 1 (10 mg/kg, p.o.); diazepam (1 mg/kg, p.o.); Compound 1+U73122; Diazepam+U73122. *p≦0.05, p** p≦0.01 significantly different compared to control group; ̂p≦0.05 ̂̂p≦0.01 significantly different compared to Compound 1 treatment alone (N=10 mice).

FIG. 6 shows that Compound 1 does not produce signs of withdrawal following a 14-day dosing period. Rats treated chronically with opioids, benzodiazepines, or SSRIs display adverse physical effects after non-precipitated withdrawal of the drugs. The potential consequences of abrupt cessation of dosing with Compound 1 was assessed following 14 days of treatment at 0, 10, 30, and 100 mg/kg/day.

FIGS. 7A and 7B show that Compound 1-induced enhancement of neurite outgrowth in rat primary cortical neurons is blocked by the PLC inhibitors D609 and Edelfosine. Results are expressed as mean±sem. ***p≦0.001 significantly different compared to inhibitor alone; ̂̂p≦0.01 ̂̂̂p≦0.001 significantly different compared to control group (N=80).

### DETAILED DESCRIPTION OF THE INVENTION

The present disclosure contemplates the treatment or prophylaxis of a disease of the central nervous system, such as mood disorders (e.g., depression) and neurodegenerative diseases. The term neurodegenerative disease encompasses a condition leading to the progressive loss of structure or function of neurons, including death of neurons. Examples of neurodegenerative diseases contemplated herein include AIDS dementia complex, adrenoleukodystrophy, alexander disease, Alpers' disease, amyotrophic lateral sclerosis, ataxia telangiectasia, Batten disease, bovine spongiform encephalopathy, Canavan disease, corticobasal degeneration, Creutzfeldt-Jakob disease, dementia with Lewy bodies, fatal familial insomnia, frontotemporal lobar degeneration. Huntington's disease, infantile Refsum disease, Kennedy's disease, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progranulin, progressive supranuclear palsy, protein aggregation, Refsum disease, Sandhoff disease, diffuse myelinoclastic sclerosis, Shy-Drager syndrome, spinocerebellar ataxia, subacute combined degeneration of spinal cord, Tabes dorsalis, Tay-Sachs disease, toxic encephalopathy, transmissible spongiform encephalopathy, and Wobbly hedgehog syndrome.

It is proposed herein that the compounds of formula (I) treat, ameliorate the symptoms of, prevent, or otherwise delay onset or development of the CNS disease or condition.

With respect to the compounds of formula (I) and other subformulae described herein, “alkyl” refers to a saturated monovalent hydrocarbon radical which may be straight chained or branched and particularly having from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like. One of ordinary skill in the art will understand that Me is methyl, Et is ethyl, and Pr is propyl.

“Aryl” refers to an unsaturated aromatic carbocyclic group having a single ring (e.g., phenyl) or multiple condensed rings (e.g., naphthyl or anthryl), particularly having from 6 to 14 carbon atoms. Examples of aryl groups include phenyl, naphthyl and the like.

“Aryloxy” refers to the group aryl-O— wherein the aryl group is as described above.

“Arylalkyl” refers to -alkylene-aryl groups preferably having from 1 to 10 carbon atoms in the alkylene moiety and from 6 to 10 carbon atoms in the aryl moiety. Such arylalkyl groups are exemplified by benzyl, phenethyl and the like.

“Arylalkoxy” refers to the group arylalkyl-O— wherein the arylalkyl group are as described above. Such arylalkoxy groups are exemplified by benzyloxy and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyl” refers to a monovalent hydrocarbon radical with at least one site of unsaturation, i.e., a carbon-carbon, sp² double bond, which may be straight chained or branched and particularly have from 2 to 10 carbon atoms and more particularly 2 to 6 carbon atoms and have at least 1 and particularly from 1-2, carbon to carbon, double bonds. An alkenyl radical includes radicals having “cis” and “trans” orientations, or alternatively, “E” and “Z” orientations. Examples include ethenyl (—CH═CH₂), n-propenyl (—CH₂CH═CH₂), iso-propenyl (—C(CH₃)═CH₂), but-2-enyl (—CH₂CH═CHCH₃), and the like.

“Alkenyloxy” refers to the group alkenyl-O— wherein the alkenyl group is as described above.

“Alkynyl” refers to a linear or branched monovalent hydrocarbon radical with at least one site of unsaturation, i.e., a carbon-carbon sp triple bond, preferably having from 2 to 10 carbon atoms and more particularly 2 to 6 carbon atoms and having at least 1, and particularly from 1-2, carbon to carbon, triple bonds. Examples of alkynyl groups include ethynyl (—C═CH), propargyl (—CH₂C═CH), pent-2-ynyl (—CH₂C≡CCH₂—CH₃), and the like.

“Alkynyloxy” refers to the group alkynyl-O— wherein the alkynyl group is as described above.

“Acyl” refers to groups H—C(O)—, alkyl-C(O)—, cycloalkyl-C(O)—, aryl-C(O)—, heteroaryl-C(O)— and heterocyclyl-C(O)—, where alkyl, cycloalkyl, aryl, heteroaryl and heterocyclyl.

“Oxyacyl” refers to groups HOC(O)—, alkyl-OC(O)—, cycloalkyl-OC(O)—, aryl-OC(O)—, heteroaryl-OC(O)—, and heterocyclyl-OC(O)—, where alkyl, cycloalkyl, aryl, heteroaryl and heterocyclyl are as described herein.

“Amino” refers to the group —NR^{A}R^{A} where each R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Aminoacyl” refers to the group —C(O)NR^{A}R^{A} where each R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Acylamino” refers to the group —NR^{A}C(O)R^{A} where each R^{A} is independently hydrogen, alkyl, cycloalkyl, aryl, heteroaryl or heterocyclyl, and where each of alkyl, cycloalkyl, aryl, heteroaryl, and heterocyclyl are as described herein.

“Acyloxy” refers to the groups —OC(O)-alkyl, —OC(O)-aryl, —C(O)O-heteroaryl, and —C(O)O-heterocyclyl, where alkyl, aryl, heteroaryl and heterocyclyl are as described herein.

“Aminoacyloxy” refers to the groups —OC(O)NR^{A}-alkyl, —OC(O)NR^{A}-aryl, —OC(O)NR^{A}-heteroaryl, and —OC(O)NR^{A}-heterocyclyl, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Oxyacylamino” refers to the groups —NR^{A}C(O)O-alkyl, —NR^{A}C(O)O-aryl, —NR^{A}C(O)O-heteroaryl, and NR^{A}C(O)O-heterocyclyl where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Oxyacyloxy” refers to the groups —OC(O)O-alkyl, —O—C(O)O-aryl, —OC(O)O-heteroaryl, and —OC(O)O-heterocyclyl, where alkyl, cycloalkyl, aryl, heteroaryl, and heterocyclyl are as described herein.

“Acylimino” refers to the groups —C(NR^{A})—R^{A} where each R^{A} is independently hydrogen, alkyl, cycloalkyl, aryl, heteroaryl or heterocyclyl, and where each of alkyl, cycloalkyl, aryl, heteroaryl, and heterocyclyl are as described herein.

“Acyliminoxy” refers to the groups —O—C(NR^{A})—R^{A} where each R^{A} is independently hydrogen, alkyl, cycloalkyl, aryl, heteroaryl or heterocyclyl, and where each of alkyl, cycloalkyl, aryl, heteroaryl, and heterocyclyl are as described herein.

“Oxyacylimino” refers to the groups —C(NR^{A})—OR^{A} where each R^{A} is independently hydrogen, alkyl, cycloalkyl, aryl, heteroaryl or heterocyclyl, and where each of alkyl, cycloalkyl, aryl, heteroaryl, and heterocyclyl are as described herein.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like. The term also includes polycyclic ring systems where the cycloalkyl ring is fused with one or more aromatic or non-aromatic carbocyclic or heterocyclic rings, such as adamantanyl, indanyl, 1,2,3,4-tetrahydronapthalenyl and the like.

“Cycloalkenyl” refers to cyclic alkenyl groups having a single cyclic ring or multiple condensed rings, and at least one point of internal unsaturation, preferably incorporating 4 to 11 carbon atoms. Examples of suitable cycloalkenyl groups include, for instance, cyclobut-2-enyl, cyclopent-3-enyl, cyclohex-4-enyl, cyclooct-3-enyl, indenyl and the like.

“Halo” or “halogen” refers to fluoro, chloro, bromo and iodo.

“Heteroaryl” refers to a monovalent aromatic heterocyclic group which fulfills the Hückel criteria for aromaticity (i.e., contains 4n+2π electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (e.g., pyridyl, pyrrolyl or N-oxides thereof or furyl) or multiple condensed rings (e.g., indolizinyl, benzoimidazolyl, coumarinyl, quinolinyl, isoquinolinyl or benzothienyl). It will be understood that for an optionally substituted heteroaryl which has one or more ring heteroatoms, the heteroaryl group can be connected to the core molecule of the compounds of the present invention, through a C—C or C-heteroatom bond, in particular a C—N bond.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium, and phosphorous within the ring. In some embodiments, the heteroatom is nitrogen. It will be understood that for an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Thio” refers to groups H—S—, alkyl-S—, cycloalkyl-S—, aryl-S—, heteroaryl-S—, and heterocyclyl-S—, where alkyl, cycloalkyl, aryl, heteroaryl and heterocyclyl are as described herein.

“Thioacyl” refers to groups H—C(S)—, alkyl-C(S)—, cycloalkyl-C(S)—, aryl-C(S)—, heteroaryl-C(S)—, and heterocyclyl-C(S)—, where alkyl, cycloalkyl, aryl, heteroaryl and heterocyclyl are as described herein.

“Oxythioacyl” refers to groups HO—C(S)—, alkylO—C(S)—, cycloalkylO—C(S)—, arylO—C(S)—, heteroarylO—C(S)—, and heterocyclylO—C(S)—, where alkyl, cycloalkyl, aryl, heteroaryl and heterocyclyl are as described herein.

“Oxythioacyloxy” refers to groups HO—C(S)—O—, alkylO—C(S)—O—, cycloalkylO—C(S)—O—, arylO—C(S)—O—, heteroarylO—C(S)—O—, and heterocyclylO—C(S)—O—, where alkyl, cycloalkyl, aryl, heteroaryl and heterocyclyl are as described herein.

“Phosphorylamino” refers to the group —NR^{A}—P(O)(R^{B})(OR^{C}) where R^{A} represents H, alkyl, cycloalkyl, alkenyl, or aryl, R^{B} represents OR^{C} or is hydroxy or amino and R^{C} is alkyl, cycloalkyl, aryl or arylalkyl, where alkyl, amino, alkenyl, aryl, cycloalkyl, and arylalkyl are as described herein.

“Thioacyloxy” refers to groups H—C(S)—O—, alkyl-C(S)—O—, cycloalkyl-C(S)—O—, aryl-C(S)—O—, heteroaryl-C(S)—O—, and heterocyclyl-C(S)—O—, where alkyl, cycloalkyl, aryl, heteroaryl, and heterocyclyl are as described herein.

“Sulfinyl” refers to groups H—S(O)—, alkyl-S(O)—, cycloalkyl-S(O)—, aryl-S(O)—, heteroaryl-S(O)—, and heterocyclyl-S(O)—, where alkyl, cycloalkyl, aryl, heteroaryl and heterocyclyl are as described herein.

“Sulfonyl” refers to groups H—S(O)₂—, alkyl-S(O)₂—, cycloalkyl-S(O)₂—, aryl-S(O)₂—, heteroaryl-S(O)₂—, and heterocyclyl-S(O)₂—, where alkyl, cycloalkyl, aryl, heteroaryl and heterocyclyl are as described herein.

“Sulfinylamino” refers to groups H—S(O)—NR^{A}—, alkyl-S(O)—NR^{A}—, cycloalkyl-S(O)—NR^{A}—, aryl-S(O)—NR^{A}—, heteroaryl-S(O)—NR^{A}—, and heterocyclyl-S(O)—NR^{A}—, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Sulfonylamino” refers to groups H—S(O)₂—NR^{A}—, alkyl-S(O)₂—NR^{A}—, cycloalkyl-S(O)₂—NR^{A}—, aryl-S(O)₂—NR^{A}—, heteroaryl-S(O)₂—NR^{A}—, and heterocyclyl-S(O)₂—NR^{A}—, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Oxysulfinylamino” refers to groups HO—S(O)—NR^{A}—, alkylO—S(O)—NR^{A}—, cycloalkylO—S(O)—NR^{A}—, arylO—S(O)—NR^{A}—, heteroarylO—S(O)—NR^{A}—, and heterocyclylO—S(O)—NR^{A}—, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Oxysulfonylamino” refers to groups HO—S(O)₂—NR^{A}—, alkylO-S(O)₂—NR^{A}—, cycloalkylO-S(O)₂—NR^{A}, arylO—S(O)₂—NR^{A}—, heteroarylO—S(O)₂—NR^{A}—, and heterocyclylO—S(O)₂—NR^{A}—, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Aminothioacyl” refers to groups R^{A}R^{A}N—C(S)—, where each R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Thioacylamino” refers to groups H—C(S)—NR^{A}—, alkyl-C(S)—NR^{A}—, cycloalkyl-C(S)—NR^{A}—, aryl-C(S)—NR^{A}—, heteroaryl-C(S)—NR^{A}—, and heterocyclyl-C(S)—NR^{A}—, where R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Aminosulfinyl” refers to groups R^{A}R^{A}N—S(O)—, where each R^{A} is independently hydrogen, alkyl, cycloalkyl, aryl, heteroaryl, or heterocyclyl, and where each of alkyl, cycloalkyl, aryl, heteroaryl and heterocyclyl is as described herein.

“Aminosulfonyl” refers to groups R^{A}R^{A}N—S(O)₂—, where each R^{A} is independently hydrogen, alkyl, cycloalkyl, aryl, heteroaryl, or heterocyclyl, and where each of alkyl, cycloalkyl, aryl, heteroaryl and heterocyclyl is as described herein.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroaryloxy, heterocyclyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

In certain embodiments, the “optionally substituted” group is halo (e.g., chloro, fluoro or bromo), —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂.

As described generally above, the present invention provides methods of administering and uses of compounds of formula (I) and pharmaceutically acceptable salts thereof:

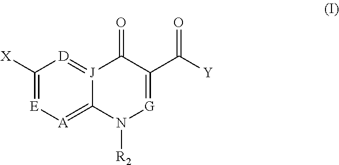

wherein A, E, G, D, J, X, Y, and R₂ are as described herein.

In some embodiments, for a compound of formula (I), Y is NR₃R₄ wherein R₃ and R₄ each independently represent H, optionally substituted alkyl, optionally substituted cycloalkyl, or together with the N-atom optionally substituted N-containing heteroaryl or optionally substituted N-containing heterocyclyl.

In some embodiments, for a compound of formula (I), Y is OR′″ (where R′″ is optionally substituted alkyl).

In some embodiments, J is C, and G is CR′, to give a compound of formula (II):

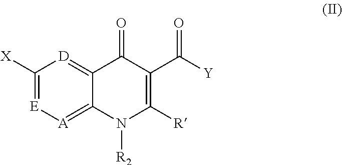

wherein A, E, D, X, R′, R₂, and Y are as described herein.

In certain embodiments, two of A, E or D are N and the other is CR′, J is C and G is CR′.

Accordingly, compounds of formula (I) contemplated herein include those represented by formulae (Ia), (Ib), and (Ic):

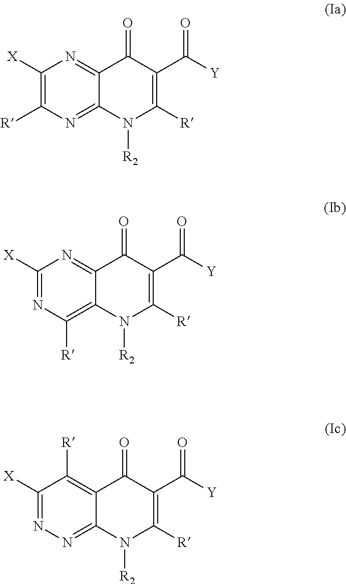

where R₂, R′, Y and X are as described above for compounds of formula (I).

In another embodiment, only one of A, E and D is N and the other two are independently CR′.

Accordingly, compounds contemplated herein include those represented by formulae (Id), (Ie) and (If):

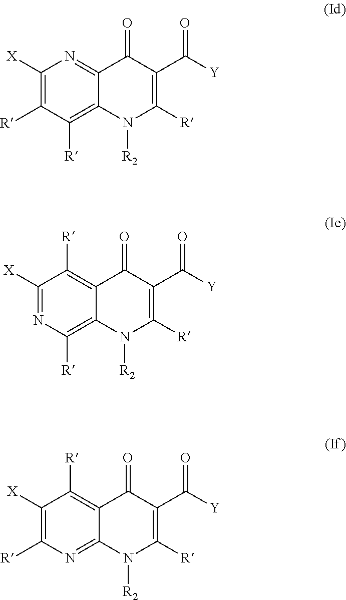

where R₂, R′, Y and X are as described above for compounds of formula (I).

In an embodiment, the compounds of the invention are represented by formula (If).

In an embodiment, the compounds of the invention are represented by formula (Ic).

In an embodiment, the compounds of the invention are represented by formula (Id).

In certain embodiments, R′ in CR′, when present, includes the following groups:

H, carboxyl, cyano, halogen, hydroxy, nitro, phosphono, phosphorylamino, phosphinyl, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, or optionally substituted thioacylamino.

In some embodiments, R′ is halogen, cyano, nitro, or amino. In certain embodiments, R′ is bromo or chloro. In some embodiments, R′ is fluoro.

In some embodiments, R′ is an optionally substituted alkyl group. In certain embodiments, R′ is an unsubstituted alkyl group. In certain embodiments, R′ is a substituted alkyl group. In certain embodiments, R′ is optionally substituted C₁-C₆ alkyl. In certain embodiments, R′ is optionally substituted C₁-C₃ alkyl. In certain embodiments, R′ is methyl or ethyl. In certain embodiments, R′ is 1-hydroxyethyl, 1-thioethyl, methoxyiminomethyl, ethoxyiminomethyl, 1-(hydroxyimino)ethyl, 1-(hydroxyimino)propyl, 1-hydrazinoethyl, 1-hydrazinopropyl, hydroxyiminomethyl, 2-oxopropyl, 2-oxobutyl, 3-oxobutyl, 3-oxopentyl, nitromethyl, 1-nitromethyl, or 2-nitroethyl. In certain embodiments, R′ is trihalomethyl. In certain embodiments, R′ is trifluoromethyl. In certain embodiments, R′ is pentahaloethyl.

In some embodiments, R′ is an optionally substituted aryl group. In certain embodiments, R′ is unsubstituted aryl. In certain embodiments, R′ is phenyl. In certain embodiments. R′ is naphthyl. In certain embodiments, R′ is substituted aryl. In certain embodiments, R′ is halophenyl (for instance, fluorophenyl), aminophenyl, carboxyphenyl, hydroxyphenyl, cyanophenyl, nitrophenyl, trihaloalkylphenyl, or alkylphenyl.

In some embodiments. R′ is an optionally substituted acyl group. In certain embodiments, R′ is unsubstituted acyl. In certain embodiments, R′ is substituted acyl. In certain embodiments, R′ is formyl, acetyl, propionyl, or benzoyl. In certain embodiments, R′ is formyl, acetyl, propionyl, or benzoyl, optionally substituted with methyl, methoxy, halogen, nitro, trifluoromethyl or cyano.

In some embodiments, R′ is a substituted or unsubstituted alkoxy group. In certain embodiments, R′ is C₁-C₆ alkoxy. In certain embodiments, R′ is C₁-C₃ alkoxy. In certain embodiments, R′ is methoxy or ethoxy. In certain embodiments, R′ is trihalomethoxy. In certain embodiments, R′ is trifluoromethoxy. In certain embodiments, R′ is dihalomethoxy.

In some embodiments, R′ is a substituted or unsubstituted oxyacyl group. In certain embodiments, R′ is C₁-C₆ alkoxycarbonyl. In certain embodiments, R′ is methoxycarbonyl, ethoxycarbonyl, propoxycarbonyl, butyloxycarbonyl, or isobutyloxycarbonyl.

In some embodiments, R′ is a substituted or unsubstituted acyloxy group. In certain embodiments, R′ is C₁-C₆ acyloxy. In certain embodiments, R′ is acetoxy or propioxy.

In some embodiments, R′ is an optionally substituted arylalkyl group. In certain embodiments, R′ is an unsubstituted arylalkyl group. In certain embodiments, R′ is benzyl. In certain embodiments, R′ is a substituted arylalkyl group. In certain embodiments, R′ is 1-hydroxybenzyl or 1-thiobenzyl.

In some embodiments, R′ is an optionally substituted sulfinyl group. In certain embodiments, R′ is alkylsulfinyl or arylsulfinyl. In certain embodiments, R′ is alkoxysulfinyl. In certain embodiments, R′ is methylsulfinyl, ethylsulfinyl, benzene sulfinyl, methoxysulfinyl, or ethoxysulfinyl. In certain embodiments, R′ is benzene sulfinyl, optionally substituted with methyl, methoxy, halogen, nitro, trifluoromethane or cyano.

In some embodiments, R′ is an optionally substituted sulfonyl group. In certain embodiments, R′ is alkylsulfonyl or arylsulfonyl. In certain embodiments, R′ is methylsulfonyl, ethylsulfonyl, or benzenesulfonyl (optionally substituted with methyl, methoxy, halogen, nitro, trifluoromethane or cyano).

In some embodiments, R′ is an optionally substituted oxyacylamino group. In certain embodiments, R′ is C₁-C₆ alkoxycarbonylamido. In certain embodiments, R′ is methoxycarbonylamido or ethoxycarbonylamido.

In some embodiments, R′ is an optionally substituted oxythioacyl group. In certain embodiments, R′ is C₁-C₆ alkoxythiocarbonyl. In certain embodiments, R′ is methoxythiocarbonyl or ethoxythiocarbonyl. In some embodiments, R′ is an optionally substituted thioacyloxy group. In certain embodiments, R′ is thionoacetoxy or thionopropionoxy.

In some embodiments, R′ is an optionally substituted sulphinylamino group. In certain embodiments, R is alkylsulfinylamino or arylsulfinylamino. In certain embodiments, R′ is methylsulfinylamino, ethylsulfinylamino, or benzenesulfinylamino. In certain embodiments, R′ is benzenesulfinylamino optionally substituted with methyl, methoxy, halogen, nitro, trifluoromethane or cyano.

In some embodiments, R′ is an amino group. In certain embodiments, R′ is alkylamino or dialkylamino. In certain embodiments, R′ is N-methylamino or N,N′-dimethylamino. In certain embodiments, R′ is a substituted amino group, such as a residue of L-valine, D-valine, L-alanine, D-alanine, aspartic acid, or alanylserine.

In certain embodiments, R′ is an optionally substituted sulphonylamino group. In certain embodiments, R′ is alkylsulfonylamino or arylsulfonylamino. In certain embodiments. R′ is C₁-C₆ alkylsulfonylamino. In certain embodiments, R′ is methylsulfonylamino, ethylsulfonylamino or benzenesulfonylamino. In certain embodiments, R′ is benzenesulfonylamino optionally substituted with methyl, methoxy, halogen, nitro, trifluoromethane or cyano.

In some embodiments, R′ is an optionally substituted thio group. In certain embodiments, R′ is a substituted thio group. In certain embodiments, R′ is alkylthio. In certain embodiments, R′ is C₁-C₆ alkylthio. In certain embodiments, R′ is thiomethyl or thioethyl. In certain embodiments, R′ is trihalomethanethio.

In some embodiments, R′ is an optionally substituted oxysulfinylamino group. In certain embodiments, R′ is alkoxysulfinylamino. In certain embodiments, R′ is methoxysulfinylamino or ethoxysulfinylamino.

In some embodiments, R′ is an optionally substituted oxysulfonylamino group. In certain embodiments, R′ is alkoxylsulfonylamino. In certain embodiments, R′ is methoxysulfonylamino or ethoxysulfonylamino.

In some embodiments, R′ is an optionally substituted alkenyl group. In some embodiments, R′ is unsubstituted alkenyl. In some embodiments, R′ is substituted alkenyl. In certain embodiments, R′ is 1-propenyl, vinyl, nitrovinyl, cyano vinyl, or trifluorovinyl or styryl. In certain embodiments R′ is styryl optionally substituted with methyl, methoxy, halogen, nitro, trifluoromethane or cyano. In certain embodiments, R′ is trihaloethenyl.

In certain embodiments, where present, CR′ is CH. In certain embodiments, all instances of CR′ are CH. Accordingly, in certain embodiments, compounds described herein are of formula:

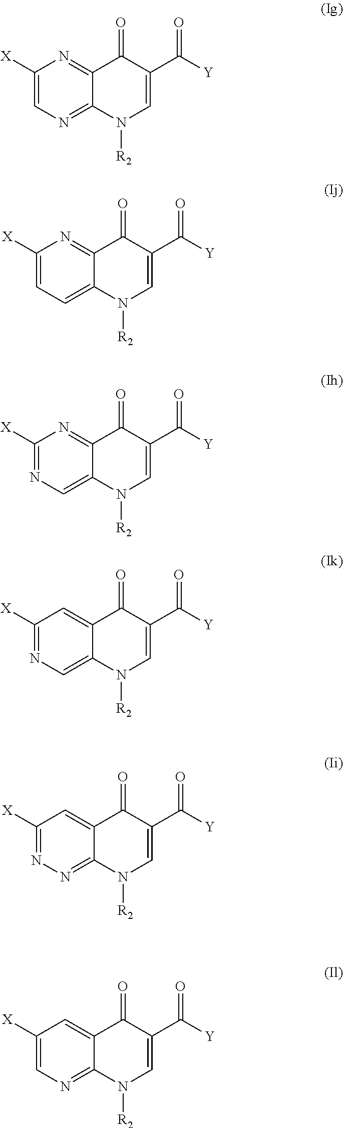

where R₂, Y and X are as defined above for compounds of formula (I).

In an embodiment, the compounds of the invention are represented by formula (Ig).

In an embodiment, the compounds of the invention are represented by formula (Ih).

In an embodiment, the compounds of the invention are represented by formula (Ii).

In an embodiment, the compounds of the invention are represented by formula (Ij).

In an embodiment, the compounds of the invention are represented by formula (Ik).

In an embodiment, the compounds of the invention are represented by formula (Il).

In certain embodiments of formula (I) and subformulae described herein, R₂ includes hydrogen, C₁₋₆ alkyl, benzyl or acetyl. In certain embodiments, R₂ is C₁₋₃ alkyl.

In certain embodiments, for a compound of formula (Ig), (Ih), (Ii), (Ij), (Ik), or (Il), R₂ is ethyl.

In certain embodiments of formula (I) and subformulae described herein, X is NR₁R″ where R″ is hydrogen, C₁₋₃ alkyl, benzyl, or acetyl. In another embodiment, X is NHR₁.

In certain embodiments of formula (I) and subformulae described herein, R₁ is optionally substituted alkyl, optionally substituted acyl, optionally substituted cycloalkyl, or optionally substituted cycloalkenyl. Substitutents include optionally substituted acyl (for instance, optionally substituted phenylacyl or optionally substituted alkyl acyl), optionally substituted aryl, halogen, COOH, NH₂, methoxy, mono or dialkyl amino or CF₃. In certain embodiments, R₁ is benzofused C₅-C₇ cycloalkyl (wherein the benzene ring may be optionally substituted). In certain embodiments, R₁ is indanyl. In certain embodiments, R₁ is 1,2,3,4-tetrahydronaphthalenyl.

In certain embodiments, the compound is a compound of formula (If) or (Il) where X is NH₂, NH(C₁-C₆ alkyl), NHC(O)C₁-C₆ alkyl, NHC(O) optionally substituted aryl, or NHbenzofused C₅-C₇ cycloalkyl (wherein the benzene group may be optionally substituted).

In certain embodiments the compound is a compound of formula (If) or (Il) wherein X is NH₂, NHC(O)C₁-C₆ alkyl, NHC(O) optionally substituted phenyl, or indanyl.

In certain embodiments of formula (I) and subformulae described herein, Y is NR³R⁴. In a particular embodiment, one of R³ and R⁴ is H and the other is optionally substituted alkyl, optionally substituted aryl, optionally substituted C₃₋₇ cycloalkyl, optionally substituted heteroaryl, or optionally substituted heterocyclyl. In other embodiments, both R₃ and R₄ are each independently optionally substituted C₁₋₃ alkyl.

In certain embodiments of formula (I) and subformulae defined herein, Y is OR′″ wherein R′″ is optionally substituted C₁₋₆ alkyl.

In further embodiments, Y is NR₃R₄, where R₃ and R₄ together with the N-atom represent an optionally substituted N-containing heteroaryl or optionally substituted N-containing heterocyclyl. In certain embodiments, NR₃R₄ form an optionally substituted N-containing heterocyclyl. In certain embodiments, NR₃R₄ form an unsubstituted N-containing heterocyclyl. In certain embodiments, NR₃R₄ form an optionally substituted 5-membered N-containing heterocyclyl. In certain embodiments, NR₃R₄ form an optionally substituted 6-membered N-containing heterocyclyl. In certain embodiments, NR₃R₄ form morpholinyl, piperidyl, piperazinyl, pyrrolidinyl, pyrazolinyl, pyrazolidinyl, imidazolinyl or indolinyl. In certain embodiments, NR₃R₄ form morpholinyl.

In certain embodiments, the compound is a compound of formula (If) or (Il) where X is NH₂, NH(C₁-C₆ alkyl), NHC(O) C₁-C₆ alkyl, NHC(O) optionally substituted aryl, or NHbenzofused C₅-C₇ cycloalkyl (wherein the benzene group may be optionally substituted), and Y is C₁₋₆ alkoxy, NH(C₁₋₆ alkyl), NH (optionally substituted aryl) and NH heterocyclyl.

In another embodiment the compound of a compound of formula (If) or (Il) wherein X is NH₂, NHC(O)C₁-C₆ alkyl, NHC(O) optionally substituted phenyl, or indanyl, and Y is C₁₋₆ alkoxy, NH(C₁₋₆ alkyl), NH (optionally substituted aryl) and NH heterocyclyl.

In another embodiment the compound is a compound of formula (If) or (Il) where X is NH₂, NHC(O)C₁-C₆ alkyl, NHC(O)(phenyl substituted 1 to 3 times independently by the group selected from halo, —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂), or NH heterocyclyl) or indanyl and Y is C₁₋₆ alkoxy, NH(C₁₋₆ alkyl), NH (phenyl substituted by the group selected from halo, —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂), or NH heterocyclyl.

Further compounds contemplated herein include those represented by formula (I′) or salts thereof

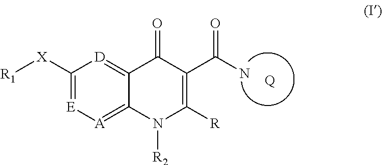

where A, E, and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N, and wherein at least one of A, E and D is N;
   X represents O or NR″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heteroaryl, optionally substituted oxysulfinyl, optionally substituted oxysulfonyl, and optionally substituted sulfinyl, optionally substituted sulfonyl);
   R represents H or optionally substituted alkyl;
   R₁ represents optionally substituted cycloalkyl, optionally substituted alkyl, optionally substituted acyl, optionally substituted aryl, optionally substituted heterocyclyl, or optionally substituted heteroaryl;
   R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
   Q represents an optionally substituted N-containing heterocyclyl or an optionally substituted N-containing heteroaryl.

In certain embodiments, two of A, E and D are N, and the other is CR′. Accordingly, compounds of formula (I′) include those represented by formulae (I′a), (I′b), and (I′c):

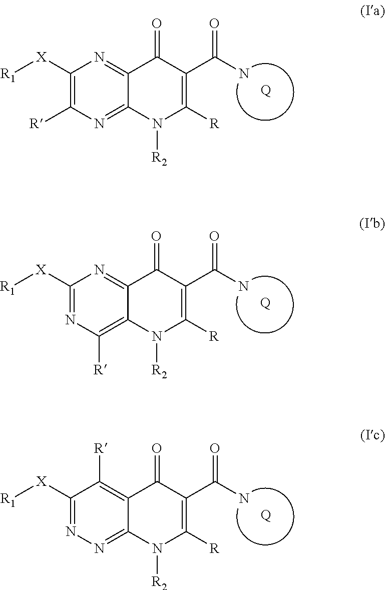

where R, R₁, Q, R′ and X are as defined herein.

In an embodiment, the compounds of the invention are represented by formula (I′a).

In an embodiment, the compounds of the invention are represented by formula (I′b).

In an embodiment, the compounds of the invention are represented by formula (I′c).

In certain embodiments, only one of A, E, and D is N, and the other two are independently CR′. Accordingly, compounds encompassed herein include those represented by formulae (I′d), (I′e), and (I′f):

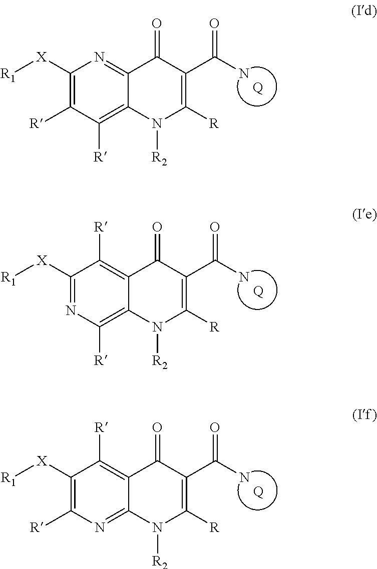

where R, R₁, Q, R′ and X are as defined herein.

In an embodiment, the compounds of the invention are represented by formula (I′d).

In an embodiment, the compounds of the invention are represented by formula (I′e).

In an embodiment, the compounds of the invention are represented by formula (I′f).

In further embodiments, compounds encompassed herein include those represented by formulae:

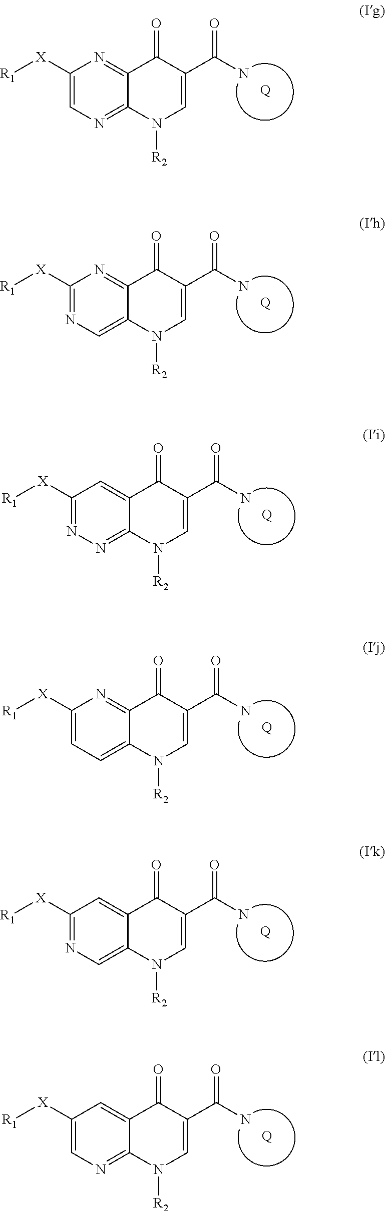

wherein X, R₁, R₂, and Q are as described herein.

In an embodiment, the compounds of the invention are represented by formula (I′g).

In an embodiment, the compounds of the invention are represented by formula (I′h).

In an embodiment, the compounds of the invention are represented by formula (I′i).

In an embodiment, the compounds of the invention are represented by formula (I′j).

In an embodiment, the compounds of the invention are represented by formula (I′k).

In an embodiment, the compounds of the invention are represented by formula (I′l).

In certain embodiments, for compounds of formula (I′g), (I′h), (I′i), (I′j), (I′k), or (I′l), R₂ is C₁-C₃ alkyl.

In certain embodiments, for compounds of formula (I′g), (I′h), (I′i), (I′j), (I′k), or (I′l), R₂ is ethyl.

In certain embodiments, Q represents optionally substituted N-containing heterocyclyl. In certain embodiments, Q represents substituted N-containing heterocyclyl. In certain embodiments, Q represents unsubstituted N-containing heterocyclyl. In certain embodiments, Q represents optionally substituted 5-membered N-containing heterocyclyl. In certain embodiments, Q represents optionally substituted 6-membered N-containing heterocyclyl. In certain embodiments, Q represents an N-containing heterocyclyl selected from morpholinyl, piperidyl, piperazinyl, pyrrolidinyl, pyrazolinyl, pyrazolidinyl, imidazolinyl and indolinyl. In certain embodiments, Q represents morpholinyl.

For compounds of formula (I′) and subformulae thereof, in certain embodiments, R is H or C₁₋₆ alkyl, more particularly R is hydrogen or methyl, and even more particularly hydrogen.

For compounds of formula (I′) and subformulae thereof, X is NR″, where R″ is hydrogen, C₁₋₃ alkyl, benzyl, or acetyl. In other embodiments, X is NH.

For compounds of formulae (I) and (I′), certain embodiments include that R₁ is optionally substituted alkyl, optionally substituted acyl, optionally substituted cycloalkyl, or optionally substituted cycloalkenyl. Substitutents include optionally substituted acyl (for instance, optionally substituted phenylacyl or optionally substituted alkyl acyl), optionally substituted aryl, halogen, COOH, NH₂, mono or dialkyl amino, or CF₃. In a certain embodiment, R₁ is —C(O)C₁-C₆ alkyl. In certain embodiments R₁ is —C(O) optionally substituted aryl. In an embodiment the aryl group is substituted 1 to 3 times independently by the group selected from halo, —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂), or NH heterocyclyl. In certain embodiments R₁ is —C(O) optionally substituted phenyl. In an embodiment the phenyl group is substituted 1 to 3 times independently with by the group selected from halo, —CN, —NO₂, —CO₂H, —CO₂C₁₋₆alkyl, —CONH₂, —CONH(C₁₋₆alkyl), —CONH(C₁₋₆alkyl)₂, —OH, hydroxyC₁₋₆alkyl, C₁₋₆alkoxy, C₁₋₆alkyl, C₁₋₆acyl, carboxyC₁₋₆alkyl, acetyl, trifluoromethyl, benzyloxy, phenyl, phenoxy, —NH₂, —NH(C₁₋₆alkyl) or —N(C₁₋₆alkyl)₂), or NH heterocyclyl. In certain embodiments, R₁ is benzofused C₅-C₇ cycloalkyl (wherein the benzene ring may be optionally substituted). In certain embodiments, R₁ is indanyl or 1,2,3,4-tetrahydronaphthalenyl.

In certain embodiments, R₁ is

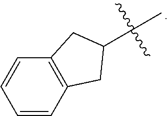 [SMILES: *C1Cc2ccccc2C1]

In certain embodiments, R₁ is

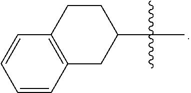 [SMILES: *C1CCc2ccccc2C1]

For certain compounds of formula (I′), R₂ includes hydrogen, C₁₋₆ alkyl, benzyl or acetyl. In certain embodiments, R₂ is C₁₋₃ alkyl. In certain embodiments, R₂ is methyl. In certain embodiments, R₂ is ethyl. In certain embodiments, R₂ is propyl.

Accordingly, in certain embodiments, the subject disclosure teaches compounds of formulae (I′f), or (I′l), or salts thereof, wherein Q represents N-containing heterocyclyl, X represents NR″ (where R″ is hydrogen, C₁₋₃ alkyl, benzyl or acetyl), R is hydrogen, R₁ represents optionally substituted cycloalkyl, optionally substituted cycloalkenyl, or —C(O)C₁₋₆ alkyl and R₂ represents C₁₋₃ alkyl.

In certain embodiments, the compound of formula (I) is a 1,8 napthyridine, where A is N, E is CH, D is CH, J is C, and G is CH.

Accordingly, in another aspect the invention provides compound of formula (I″f)

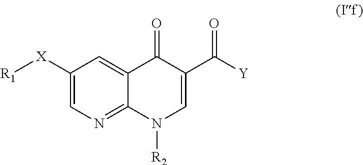

wherein variables X, R₁, R₂ and Y are as described herein.

In relation to compounds of formula (I″f), one or more, a subset, or a combination of the following definitions may apply:
Y is —OC₁-C₆ alkyl, or NR₃R₄ (where R₃ is independently H or C₁-C₆ alkyl and R₄ is C₁-C₆ alkyl), or
R₃ and R₄ together with the N-atom form an optionally substituted N-containing heteroaryl or optionally substituted N-containing heterocyclyl;
R₁X is —NH₂, —NH(C₁-C₆ alkyl), —N(C₁-C₆ alkyl)₂, —NH(benzofused C₅-C₇ cycloalkyl), —NHCO(C₁-C₆ alkyl) or —NHCO (optionally substituted aryl); and
R₂ is C₁-C₃ alkyl.

In certain embodiments, R₁X is —NHCO(C₁-C₆ alkyl), —NH(benzofused C₅-C₇ cycloalkyl) or —NHCO (optionally substituted phenyl). In certain embodiments, R₁X is

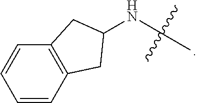 [SMILES: *NC1Cc2ccccc2C1]

In certain embodiments, R₁X is

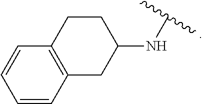 [SMILES: *NC1CCc2ccccc2C1]

Representative compounds of the present invention include:

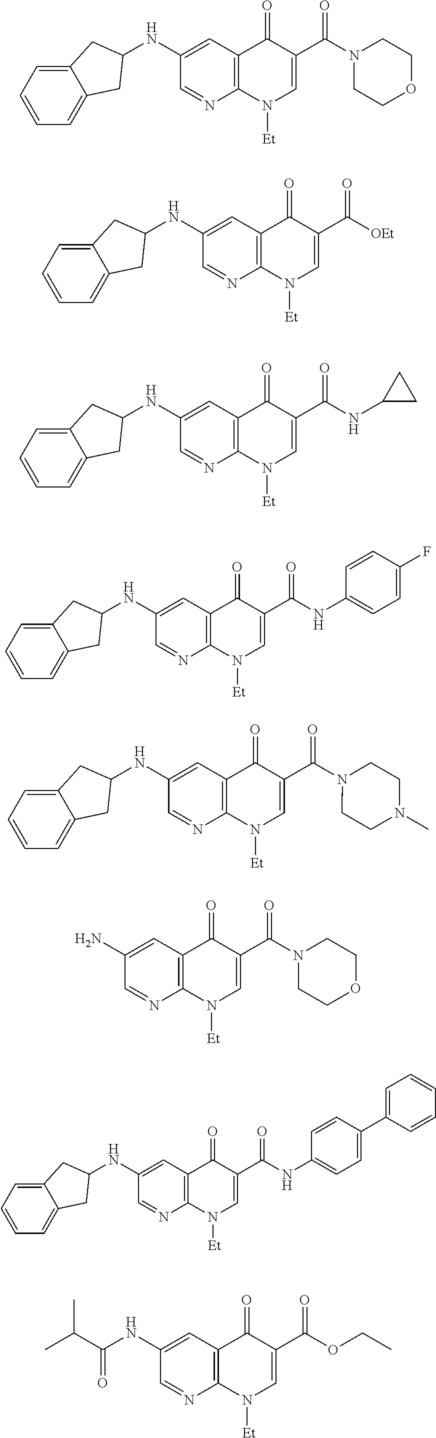

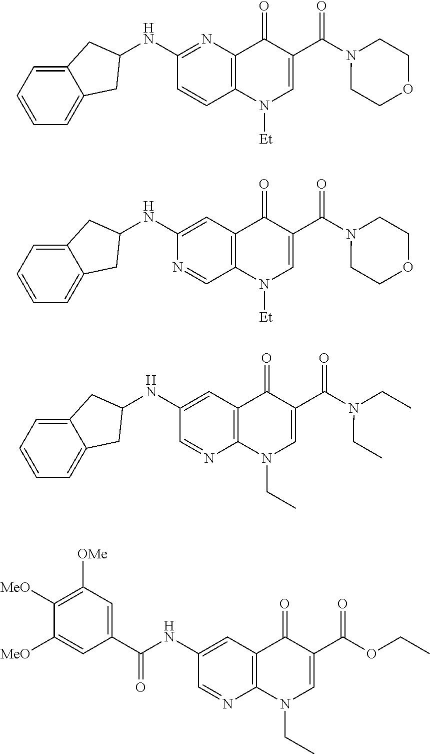

The compounds of the present invention can be prepared according to Scheme 1 below:

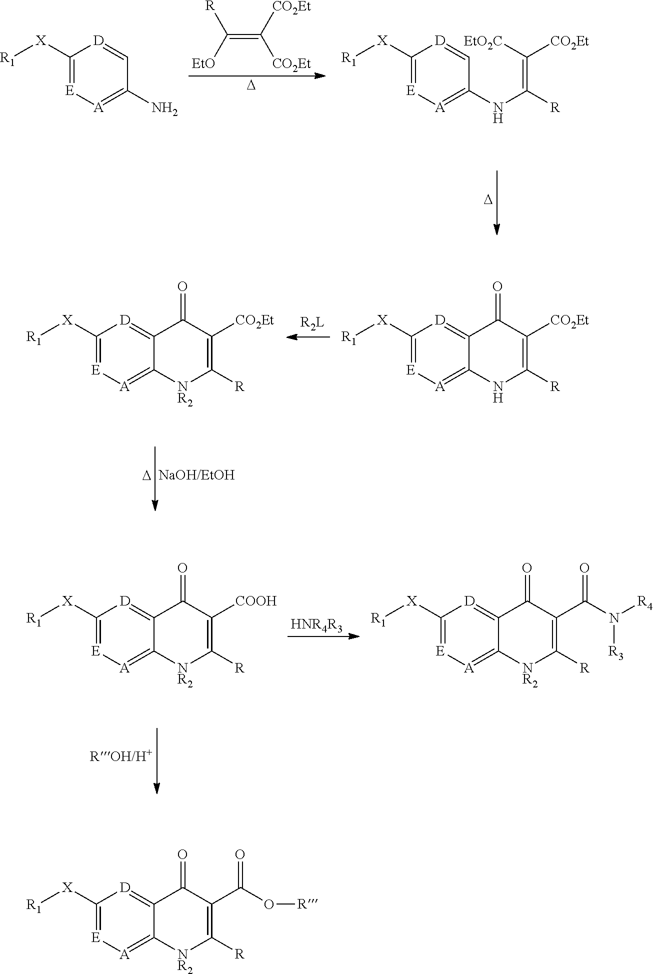

In the above Scheme, only one of A, E, or D is N. Other options, however, apply and are encompassed herein.

As shown in Scheme 1 an amino substituted N-containing heteroaryl (e.g., a 2-substituted-5-amino-pyridine) may be heated in the presence of a diethyl ethoxymethylene malonate in a suitable solvent (e.g., diethyl ether) to afford the desired diethyl aminomethylene malonate.

This product may then be cyclised at temperatures above 200° C. (for instance in diphenyl ether) to afford the corresponding ring closed product (where Y is OEt). Hydrolysis of the ethyl ester under standard conditions may afford the corresponding carboxylic acid. Alternatively, where it is desired to make compounds where R₂ is other than H, the ring closed product may be reacted with a suitable electrophilic group (e.g., alkylation with an alkylhalide) under standard conditions.

Coupling of the acid with HNR₄R₃ may be achieved under typical peptide coupling conditions. For example, the carboxylic acid can be initially converted to an activated ester with ethyl chloroformate or HBTU in the presence of a suitable non-nucleophilic base (e.g. triethylamine, Hünig's base, etc.).

Alternatively, other groups where Y is OR′″ may be produced by standard ester forming methodology with an alcohol (R′″OH) and suitable acid.

Another approach to the compounds of the present invention is depicted in Scheme 2:

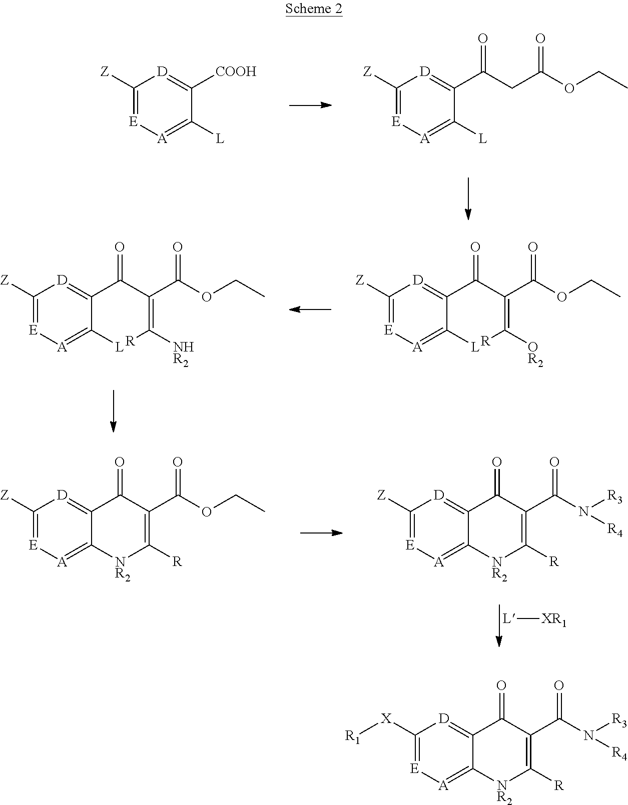

As shown in Scheme 2, a carboxy-substituted N-containing heteroaryl (e.g., a 2,5-disubstituted nicotinic acid) may be converted to the malonate ester by reaction with thionyl chloride and potassium ethyl malonate under standard conditions. The L group depicted in Scheme 2 represents any suitable leaving group which may be halogen, methoxy, tosylate, mesylate, etc. The malonate ester may be reacted with triethylorthoformate in acetic acid followed by the addition of a nucleophilic amine (HNR₂) to afford the ethylene amine which may be subsequently cyclised or be promoted to cyclise (eg in the presence of a mild base (e.g. K₂CO₃)) to afford the ring closed product. Addition of the XR₁ group may be accomplished by nucleophilic substitution chemistry with an effective nucleophilic e.g. ^{O}NHR₁ or ^{O}OR₁ or may be introduced using palladium catalysed coupling chemistry. Accordingly, Z may be an oxygen based leaving group (or precursor thereof) such as a tosylate or mesylate, or a halogen for instance, Cl, Br, or I.

In Scheme 2, Z may alternatively be NO₂. In the final stages of the synthesis (and preferably after the ring closure step) the NO₂ group may be reduced to NH₂ with the use of, for instance, Raney nickel/H₂. The corresponding NH₂ group may be reacted with RL′ (L′ is a leaving group) to produce compounds where —XR₁ is —NHR₁.

It would be appreciated then that the introduction of the X—R₁ group may take place at any convenient stage during the synthetic process and that this applies to both the strategies depicted in Schemes 1 and 2.

The preparation of di- and tri-substituted N-containing heteroaryls as starting materials in the above synthetic procedures may be accomplished using conventional chemistry (see for instance, D. T. Davies, Aromatic Heterocyclic Chemistry, 1993, Oxford Press, New York). Many such starting compounds have also been reported in the literature.

Other compounds of formulae I and I′ can be prepared by the addition, removal or modification of existing substituents. This could be achieved by using standard techniques for functional group inter-conversion that are well known in the industry, such as those described in “Comprehensive organic transformations: a guide to functional group preparations” by Larock R. C., New York, VCH Publishers, Inc. 1989.

Examples of functional group inter-conversions are: —C(O)NR*R** from —CO₂CH₃ by heating with or without catalytic metal cyanide, e.g. NaCN, and HNR*R** in CH₃OH; —OC(O)R from —OH with e.g., ClC(O)R in pyridine; —NC(S)NR*R** from —NHR with an alkylisothiocyanate or thiocyanic acid; —NRC(O)OR* from —NHR with alkyl chloroformate; —NRC(O)NR*R** from —NHR by treatment with an isocyanate, e.g. HN═C═O or RN═C═O; —NRC(O)R* from —NHR by treatment with ClC(O)R* in pyridine; —C(═NR)NR*R** from —C(NR*R**)SR with H₃NR⁺OAc⁻ by heating in alcohol; —C(NR*R**)SR from —C(S)NR*R** with R—I in an inert solvent, e.g. acetone; —C(S)NR*R** (where R* or R** is not hydrogen) from —C(S)NH₂ with HNR*R**; —C(═NCN)—NR*R** from —C(═NR*R**)—SR with NH₂CN by heating in anhydrous alcohol, alternatively from —C(═NH)—NR*R** by treatment with BrCN and NaOEt in EtOH; —NR—C(═NCN)SR from —NHR* by treatment with (RS)₂C═NCN; —NR**SO₂R from —NHR* by treatment with ClSO₂R by heating in pyridine; —NR*C(S)R from —NR*C(O)R by treatment with Lawesson's reagent [2,4-bis(4-methoxyphenyl)-1,3,2,4-dithiadiphosphetane-2,4-disulfide]; —NRSO₂CF₃ from —NHR with triflic anhydride and base, —CH(NH₂)CHO from —CH(NH₂)C(O)OR* with Na(Hg) and HCl/EtOH; —CH₂C(O)OH from —C(O)OH by treatment with SOCl₂ then CH₂N₂ then H₂O/Ag₂O; —C(O)OH from —CH₂C(O)OCH₃ by treatment with PhMgX/HX then acetic anhydride then CrO₃; R—OC(O)R* from RC(O)R* by R**CO₃H; —CCH₂OH from —C(O)OR* with Na/R*OH; —CHCH₂ from —CH₂CH₂OH by the Chugaev reaction; —NH₂ from —C(O)OH by the Curtius reaction; —NH₂ from —C(O)NHOH with TsCl/base then H₂O; —CHC(O)CHR from —CHCHOHCHR by using the Dess-Martin Periodinane regent or CrO₃/aqH₂SO₄/acetone; —C₆H₅CHO from —C₆H₅CH₃ with CrO₂Cl₂; —CHO from —CN with SnCl₂/HCl; —CN from —C(O)NHR with PCl₅; —CH₂R from —C(O)R with N₂H₄/KOH.

One of ordinary skill in the art will appreciate that the synthetic methods, as described herein, may necessitate a variety of protecting groups. By the term “protecting group”, as used herein, it is meant that a particular functional moiety, e.g., O, S, or N, is temporarily blocked so that a reaction can be carried out selectively at another reactive site in a multifunctional compound. In certain embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroactate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethylenamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pmc), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. O., Eds., John Wiley & Sons, New York: 1999, the entire contents of which are hereby incorporated by reference.

In studies conducted by the inventors it has been demonstrated that compounds disclosed herein enhance neurite outgrowth in primary cortical neurons. In certain embodiments, a compound that enhances neurite outgrowth is a compound that increases neurite outgrowth by at least 5% (e.g., at least 10%, at least 20%, at least 50%, or more in comparison to a control) in a neurite outgrowth assay, for example a neurite outgrowth assay described herein. In certain embodiments, it has been demonstrated that compounds disclosed herein enhance neurite outgrowth in primary cortical neurons which contain TrkB but do not appear to have any significant effect in PC12 cells which lack the TrkB receptor. The expression of TrkB and its ligand BDNF in limbic brain regions including the hippocampus have a critical role in the pathology of mood (affective) disorders and neurodegeneration. There are several lines of evidence to support a role for BDNF, the most abundant neurotrophin in the brain, in the action of antidepressant compounds. Exposure to stress, which is associated with the onset of many mood disorders, has consistently been shown to decrease hippocampal neurotrophin expression, in particular BDNF, while chronic antidepressant administration and/or electro-convulsive therapy, increases the expression of BDNF and its receptor TrkB, in the brain. Recent studies have also demonstrated that infusion of BDNF directly into the hippocampus produces an antidepressant-like effect in animal behavioural models of depression. In relation to the antidepressant effects of the compounds this was confirmed by the rat forced swim test (Porsolt et al, 1978, Eur J. Pharma, 47, 379-391). Furthermore, several lines of research have directly linked the depression and neurogenesis. Decreased hippocampal neurogenesis has been observed in the brains of depressed patients in post mortem studies. In addition, it has been demonstrated that when hippocampal neurogenesis is prevented, using irradiation techniques for example, antidepressants such as SSRI's and SNRI's are no longer active. The time taken for neurogenesis to occur has been proposed to account for the delay that occurs, of 2-4 weeks, before the beneficial effects of current antidepressant therapies are experienced.

Evidence is accumulating for the neurotrophic and neuroprotective effects of other psychotropic agents such as mood stabilizers, antidepressants, and antipsychotics. They also promote neurogenesis and are protective in models of neurodegenerative disease and ischemia. These effects are achieved by activation of particular intracellular signaling pathways and up-regulation of the expression of neurotrophic/neuroprotective molecules such as BDNF, NGF, Bcl-2 and AKT.

Taught herein, therefore, is the use of a compound of formula (I) or (I′), or a subformula thereof described herein, or a pharmaceutically acceptable salt thereof, in the manufacture of a medicament for treating or preventing mood disorders or neurodegenerative diseases, in a subject in need thereof.

Also provided herein is a method of treating or preventing mood disorders or neurodegenerative diseases comprising the administration of an effective amount of at least one compound of formulae (I) or (I′), or a subformula thereof described herein, or a pharmaceutically acceptable salt thereof, to a subject in need thereof.

As used herein mood disorders are broadly recognized and clearly defined by the relevant DSM-IV-TR (Diagnostic and Statistical Manual of Mental Disorders, 4^{th} Edition, Text Revision) criteria. Thus, there are depressive disorders, of which the best known and most researched is major depressive disorder (MDD) commonly called clinical depression or major depression, and bipolar disorder (BD), formerly known as manic depression and characterized by intermittent episodes of mania or hypomania, usually interlaced with depressive episodes.

Persons of skill in the art would be familiar with the lag period of traditional antidepressant medications, and with the heightened anxiety produced by the newer generation antidepressants, including SSRI's, SNRI's and NRI's in the early stages of treatment before the antidepressant effects are seen (within 2-4 weeks). Thus, in certain embodiments, the compounds described herein can be administered to a subject in need thereof as a substitute or replacement for traditional antidepressant medication. In other embodiments, there is provided a method for treating or preventing depression in a subject, the method including the step of administering to said subject a compound of formula (I) or (I′), or a subformula thereof described herein, or a pharmaceutically acceptable salt thereof, in the absence of adjunct antidepressant therapy.

Replacing traditional antidepressant medication with the present compounds can be advantageous, particularly where the traditional medication is associated with one or more adverse effects (e.g., anxiety, nausea, headaches, erectile dysfunction, early-onset suicidal tendencies, etc). Examples of traditional antidepressant medication, would be known to those skilled in the art and include, but are not limited to, selective serotonin re-uptake inhibitors (SSRI), serotonin/noradrenalin re-uptake inhibitors, selective noradrenalin re-uptake inhibitors, monoamine oxidase inhibitors, tricyclic antidepressants, lithium and other mood stabilisers, atypical antidepressants, and hormones such as estrogen or progestogen.

In other embodiments, the present compounds are administered to a subject in need thereof, together with traditional antidepressants for a period of about 2-4 weeks, to address the symptoms of depression, with the option of discontinuing treatment with the present compounds whilst continuing with the traditional therapy. In other embodiments, the subject is treated with both the present compounds and one or more traditional antidepressant medication (administered sequentially or in combination) for the duration of the treatment period. Such combination therapy may be particularly useful, for example, where the combination of the present compounds and the one or more traditional antidepressant medication provide relief from depression in the acute lag phase of the treatment period and/or where an additive or synergistic antidepressant therapeutic effect is desired.

In some embodiments, a subject according to the methods of the present invention does not suffer from an anxiety disorder. In certain embodiments, a subject does not suffer from a phobia. In certain embodiments, a subject does not suffer from one or more of agoraphobia, agoraphobia without history of panic disorder, animal phobia, and social phobia. In certain embodiments, a subject does not suffer from one or more of obsessive-compulsive disorder, stress disorders including post-traumatic and acute stress disorder, and substance-induced anxiety disorder. In certain embodiments, a subject does not suffer from generalized anxiety disorder. In certain embodiments, a subject does not suffer from social anxiety disorder.

In some embodiments, a subject according to the methods of the present invention does not suffer from one or more of neuroses, convulsions, migraine, depressive disorder, bipolar disorder, psychotic disorder, neurodegeneration arising from cerebral ischemia, attention deficit hyperactivity disorder, Tourette's syndrome, speech disorder, and disorders of circadian rhythm. In certain embodiments, a subject does not suffer from one or more of single-episode or recurrent major depressive disorder, dysthymic disorder, bipolar I or bipolar II manic disorder, and cyclothymic disorder. In certain embodiments, a subject does not suffer from schizophrenia. In certain embodiments, a subject does not suffer from stuttering.

In some embodiments, a subject according to the methods of the present invention does not suffer from one or more of pain or nociception, emesis, eating disorder, premenstrual syndrome, muscle spasm or spasticity, hearing disorder, urinary incontinence, and the effects of substance abuse or dependency. In certain embodiments, a subject does not suffer from one or more of acute emesis, delayed emesis, anticipatory emesis, emesis induced by chemotherapy or radiation, motion sickness, and post-operative nausea and vomiting. In certain embodiments, a subject does not suffer from anorexia nervosa or bulimia nervosa. In certain embodiments, a subject does not suffer from tinnitus or age-related hearing impairment. In certain embodiments, a subject does not suffer from alcohol withdrawal. In some embodiments, a subject does not suffer from Alzheimer's disease.

In some embodiments, a subject according to the methods of the present invention does not display one or more symptoms, e.g., one, two, three, four, five, six, seven, eight, nine, or ten symptoms of one or more of the following diseases or conditions: anxiety disorders, such as panic disorder with or without agoraphobia, agoraphobia without history of panic disorder, animal and other phobias including social phobias, obsessive-compulsive disorder, stress disorders including post-traumatic and acute stress disorder, and generalized or substance-induced anxiety disorder; neuroses; convulsions; migraine; depressive or bipolar disorders, for example single-episode or recurrent major depressive disorder, dysthymic disorder, bipolar I and bipolar II manic disorders, and cyclothymic disorder; psychotic disorders including schizophrenia; neurodegeneration arising from cerebral ischemia; attention deficit hyperactivity disorder; Tourette's syndrome; speech disorders, including stuttering; and disorders of circadian rhythm, e.g. in subjects suffering from the effects of jet lag or shift work; pain and nociception; emesis, including acute, delayed and anticipatory emesis, in particular emesis induced by chemotherapy or radiation, as well as motion sickness, and post-operative nausea and vomiting; eating disorders including anorexia nervosa and bulimia nervosa; premenstrual syndrome; muscle spasm or spasticity, e.g., in paraplegic patients; hearing disorders, including tinnitus and age-related hearing impairment; urinary incontinence; and the effects of substance abuse or dependency, including alcohol withdrawal; dementing conditions; and Alzheimer's disease. Depression relapse can also occur in patients treated with traditional antidepressant medication. Many such compounds are administered for anywhere from months to years and a reduction in efficacy is often seen with such long-term use, leading to significant continuing depression and dysfunction. Depression relapse may be sudden onset for some patients, while for others it might be evident as a gradual decline in mood and function, which diminishes over time as the patient approaches the state of relapse. Thus, patients who experience sudden onset of depression relapse or a gradual depression relapse would benefit from the methods disclosed herein, as the present compounds of formula (I) or (I′), or a subformula thereof described herein, can offset the diminishing effect of traditional antidepressant therapy. Thus, the use of the present compounds may prevent or partly alleviate depression relapse often seen in patients taking traditional antidepressant medication.

Thus, in an embodiment, there is provided a method for treating or preventing relapse in a subject receiving antidepressant therapy, the method including the step of administering to said subject a compound of formula (I) or (I′), or a subformula thereof described herein, or a pharmaceutically acceptable salt thereof.

The traditional antidepressant therapies that are associated with potential depression relapse in a subject would be known to those skilled in the art. Examples include, but are not limited to, dosage increases, alternative SSRIs or SNRIs, and non-SSRI antidepressants such as noradrenaline re-uptake inhibitors, monoamine oxidase inhibitors, tricyclic antidepressants, lithium and other mood stabilisers, atypical antidepressants and hormones such as estrogen and progestogen, also referred to herein as “second antidepressant compounds”.

“Treat”, “treating” or “treatment” with regard to a disorder or disease refers to alleviating or abrogating the cause and/or the effects of the disorder or disease. As used herein, the terms “treat”, “treatment” and “treating” refer to the reduction or amelioration of the progression, severity and/or duration of condition, or the amelioration of one or more symptoms (e.g., one or more discernable symptoms) of said condition (i.e., “managing” without “curing” the condition), resulting from the administration of one or more therapies (e.g., one or more therapeutic agents such as a compound or composition of the invention). In specific embodiments, the terms “treat”; “treatment” and “treating” refer to the amelioration of at least one measurable physical parameter of a condition described herein. In other embodiments the terms “treat”, “treatment” and “treating” refer to the inhibition of the progression of a condition described herein, either physically by, e.g., stabilization of a discernable symptom or physiologically by, e.g., stabilization of a physical parameter, or both.

The desired therapeutic activity, or effect, will typically depend on the condition being treated. For example, where the subject is being treated for depression, the therapeutic effect may be a reduction in at least one clinical symptom of depression, including, but not limited to, cognitive impairment, loss of appetite, mood or inactivity.

The term “preventing” as used herein refers to administering a medicament beforehand to avert or forestall the appearance of one or more symptoms of a disease or disorder. The person of ordinary skill in the medical art recognizes that the term “prevent” is not an absolute term. In the medical art it is understood to refer to the prophylactic administration of a drug to substantially diminish the likelihood or seriousness of a condition, or symptom of the condition and this is the sense intended in this disclosure. The Physician's Desk Reference, a standard text in the field, uses the term “prevent” hundreds of times. As used therein, the terms “prevent”, “preventing” and “prevention” with regard to a disorder or disease, refer to averting the cause, effects, symptoms or progression of a disease or disorder prior to the disease or disorder fully manifesting itself.

In certain embodiments, the present compounds of formula (I) or (I′), or a subformula thereof described herein, or a pharmaceutically acceptable salt thereof, are administered to said subject sequentially (i.e., before or after) or in combination with the second antidepressant compound (i.e., with existing antidepressant therapy).

In addition, the present compounds have the further added advantage in that they are free of sedative side effects which may adversely affect a patient's quality of life.

Sudden discontinuation of antidepressant medication may produce withdrawal effects caused by physical dependence on the drug. Compounds can be evaluated for physical dependence in a simple animal model where, following a period of chronic dosing (e.g., for 14-20 days), the study drug is stopped and measurements of food intake, body weight and body temperature are taken over the next 5 days. The symptoms of abrupt discontinuation of the drug are manifest as significantly reduced appetite, weight loss and drop in body temperature. This model is suitable for detecting the effects across a broad range of drug classes including opiates, antidepressants and benzodiazepines. Abrupt withdrawal of the present compounds tested did not produce any changes in these parameters indicating that the compounds do not produce physical dependence and supporting their suitability for chronic use to treat mood disorders such as depression. The compounds encompassed herein may also be used as a combination therapy, e.g. combining the treatment with other antidepressants such as benzodiazepines (e.g., alprazolam, diazepam, lorazepam, elonezepam), selective serotonin re-uptake inhibitors (SSRI) (e.g. citalopram, dapoxetine, escitalopram, fluoxetine, fluvoxamine, indalpine, paroxetine, sertraline, zimelidine, vilaxodone), serotonin norepinephrine reuptake inhibitors (SNRI) (e.g. venlafaxine, duloxetine, desvenlafaxine, milnacipran), monoamine oxidase inhibitors (e.g. phenelzine, moclobemide), tricyclic antidepressants (e.g. trimipramine, imipramine), tetracyclic antidepressants (e.g. mertazepine, maprotiline), mood stabilisers (e.g. lithium, sodium valproate, valproic acid), atypical antidepressants (e.g. bupropion), acetylcholinesterase inhibitors (e.g. donepezil, galantamine, rivastigmine), atypical antipsychotics (e.g. risperidone, aripiprizole, quetiapine, olanzapine), and hormones such as estrogen and progestogen By “treating” includes prophylaxis which encompass preventing or delaying onset or progression of a CNS disease.

It will thus be understood that the compounds herein can be used in the treatment of any disease state or condition which may be ameliorated by enhancement of neurite outgrowth.

The present disclosure teaches a disease, condition, state disorder or other adverse manifestation including trauma of the CNS such as the development or progression of a neurodegenerative disease. Examples of neurodegenerative diseases contemplated herein include AIDS dementia complex, adrenoleukodystrophy, Alexander disease, Alpers' disease, amyotrophic lateral sclerosis, ataxia telangiectasia, Batten disease, bovine spongiform encephalopathy, Canavan disease, corticobasal degeneration, Creutzfeldt-Jakob disease, dementia with Lewy bodies, fatal familial insomnia, frontotemporal lobar degeneration, Huntington's disease, infantile Refsum disease, Kennedy's disease, Krabbe disease, Lyme disease, Machado-Joseph disease, mild cognitive impairment, multiple sclerosis, multiple system atrophy, neuroacanthocytosis, Niemann-Pick disease, Parkinson's disease, Pick's disease, primary lateral sclerosis, progranulin, progressive supranuclear palsy, protein aggregation, Refsum disease, Sandhoff disease, diffuse myelinoelastic sclerosis, Shy-Drager syndrome, spinocerebellar ataxia, subacute combined degeneration of spinal cord, Tabes dorsalis, Tay-Sachs disease, toxic encephalopathy, transmissible spongiform encephalopathy, and Wobbly hedgehog syndrome.

In a certain embodiment, the neurodegenerative disease is selected from the group consisting of: multiple sclerosis, Parkinson's disease, amyotrophic lateral sclerosis, and Huntington's disease.

In a certain embodiment, the neurodegenerative disease is multiple sclerosis. In another embodiment, the neurodegenerative disease is Parkinson's disease. In another embodiment, the neurodegenerative disease is amyotrophic lateral sclerosis. In another embodiment, the neurodegenerative disease is Huntington's disease.

Also contemplated herein is a sub-threshold disease, condition, state, disorder or trauma. In an embodiment, the disease, condition, state, disorder or trauma is defined by its symptoms. Hence, the compounds contemplated herein are useful in ameliorating the symptoms of a disease, condition, state, disorder or trauma of the CNS. By “trauma” this includes stroke, brain haemorrhage, or another condition or event of the systemic vasculature which affects the CNS. The symptoms of a disease, condition, state, disorder or trauma of the CNS would be familiar to those skilled in the art. Examples of such symptoms include mood disorders, such as depression. Thus, in certain embodiments, the compounds herein are used in the treatment of depression attributed to (or associated with) a neurodegenerative disease in the subject.

The compounds encompassed herein may also be used as therapy, e.g. combining the treatment with other neurodegenerative treatments, such as acetylcholinesterase inhibitors (e.g. Aricept, Exelon), and treatments for multiple sclerosis (e.g. Avonex, Betaseron, Copaxone, Tysabri, Gilenya).

In an embodiment, the neurodegenerative disease is not Alzheimer's disease. In another embodiment, the neurodegenerative disease is not a neurodegenerative disease arising from cerebral ischemia.

The compounds enclosed herein are administered to the subject in a treatment effective amount. In some embodiments, a treatment effective amount is a therapeutically effective amount or a prophylactically effective amount. The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Roughly, prophylactic measures are divided between primary prophylaxis (to prevent the development of a disease or symptom) and secondary prophylaxis (whereby the disease or symptom has already developed and the patient is protected against worsening of this process).

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages lie within the range of about 0.1 ng per kg of body weight to 1 g per kg of body weight per dosage. The dosage may be in the range of 1 g to 1 g per kg of body weight per dosage, such as is in the range of 1 mg to 1 g per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 100 mg per kg of body weight per dosage, such as up to 50 mg per body weight per dosage.

In an embodiment, the method comprises administering to a subject in need thereof the present compound in a dosage to provide an effective amount in vivo that will enhance neurite outgrowth (neurogenesis), including, but not limited to the acute stages of treatment (e.g., within 1, 2, 3, or 4 weeks from the commencement of treatment). In an embodiment, an effective amount in vivo has an in vitro equivalent concentration that is sufficient to increase neurite outgrowth by at least 5%, at least 10%, at least 20%, or at least 50% in a neurite outgrowth assay, for example, a neurite outgrowth assay described herein. Methods of determining an in vitro equivalent concentration of the present compounds would be familiar to the skilled artisan. For example, at from about 10 minutes to 60 minutes after administration of the present compounds to a subject, a blood sample is taken and assayed by HPLC, ELISA, gas chromatography or by other suitable assay to determine the concentration per ml of blood. The approximate same concentration can then be used in an in vitro assay at a range of 30%. In another embodiment, when the present compound is found to stimulate neurite outgrowth in vitro (as compared to a control), an approximate in vivo effective amount can be determined for a subject by extrapolating the in vitro concentration to an in vivo equivalent. Factors such as the weight of the subject, the appropriate blood volume of the subject and the appropriate rate of diffusion of the present compound across the blood-brain barrier may be used to extrapolate an in vivo effective amount and hence the appropriate dosage amount that would give rise to said in vivo effective amount.

Thereafter, treatment with the present compounds may be continued throughout the treatment period or it may be ceased or replaced with traditional therapeutic compounds. Methods of determining the effective amount of the present compounds that is required for enhancing neurite outgrowth (neurogenesis) in vivo would be familiar to those skilled in the art. For example, enhancement of neurogenesis can be determined by measuring a symptom of the CNS disorder including, but not limited to, cognitive impairment, degree and frequency of seizures or tremors, motordysfunction, headaches and mood (e.g., degree of happiness).

The terms “administer”, “administering” or “administration” in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

Suitable dosage amounts and dosing regimens can be determined by the attending physician and may depend on the particular condition being treated, the severity of the condition as well as the general age, health and weight of the subject. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The active ingredient may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When co-administered with other agents, e.g. when co-administered with another anti-anxiety or anti-depressant medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.01 to about 10,000 mg/kg body weight/day, about 0.01 to about 5000 mg/kg body weight/day, about 0.01 to about 3000 mg/kg body weight/day, about 0.01 to about 1000 mg/kg body weight/day, about 0.01 to about 500 mg/kg body weight/day, about 0.01 to about 300 mg/kg body weight/day, about 0.01 to about 100 mg/kg body weight/day.

When “combination therapy” is employed, an effective amount can be achieved using a first amount of a compound of Formula I or a pharmaceutically acceptable salt thereof, and a second amount of an additional suitable therapeutic agent.

In certain embodiments, the compound of formula (I) as described herein, or a pharmaceutically acceptable salt thereof, and the additional therapeutic agent are each administered in an effective amount (i.e., each in an amount which would be therapeutically effective if administered alone). In other embodiments, the compound of formula (I) as described herein, or a pharmaceutically acceptable salt thereof, and the additional therapeutic agent are each administered in an amount which alone does not provide a therapeutic effect (a sub-therapeutic dose). In yet other embodiments, the compound of formula (I) as described herein, or a pharmaceutically acceptable salt thereof can be administered in an effective amount, while the additional therapeutic agent is administered in a sub-therapeutic dose. In still other embodiments, the compound of formula (I) as described herein, or a pharmaceutically acceptable salt thereof, can be administered in a sub-therapeutic dose, while the additional therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order. When co-administration involves the separate administration of the first amount of a compound of formula (I) as described herein, or a pharmaceutically acceptable salt thereof, and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of formula (I) as described herein, or a pharmaceutically acceptable salt thereof, and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as a compound described herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

Examples of therapeutic agents that may be combined with a compound of this disclosure, either administered separately or in the same pharmaceutical composition, include, but are not limited to muscle relaxants, anticonvulants, hypnotics, anaesthetics, analgesics, cholinergics, antidepressants, mood stabilisers, anxiolytics, etc.

In an embodiment, the second therapeutic agent is a SSRI selected from the following:
citalopram (Celexa, Cipramil, Cipram, Dalsan, Recital, Emocal, Sepram, Seropram, Citox, Cital)
dapoxetine (Priligy)
escitalopram (Lexapro, Cipralex, Seroplex, Esertia)
fluoxetine (Prozac, Fontex, Scromex, Scronil, Sarafem, Ladose, Motivest, Flutop, Fluctin (EUR), Fluox (NZ), Depress (UZB), Lovan (AUS), Prodep (IND))
fluvoxamine (Luvox, Fevarin, Faverin, Dumyrox, Favoxil, Movox)
paroxetine (Paxil, Seroxat, Sereupin, Aropax, Deroxat, Divarius, Rexetin, Xetanor, Paroxat, Loxamine, Deparoc)
sertraline (Zoloft, Lustral, Serlain, Asentra)
vilazodone (Viibryd)

In another embodiment the second therapeutic agent is a tetracyclic antidepressant (TeCA) selected from the group consisting of:
Amoxapine (Amokisan, Asendin, Asendis, Defanyl, Demolox, Moxadil)
Maprotiline (Deprilept, Ludiomil, Psymion)
Mazindol (Mazanor, Sanorex)
Mianserin (Bolvidon, Depnon, Norval, Tolvon)
Mirtazapine (Remeron, Avanza, Zispin, Miro)
Setiptiline (Tecipul)

In another embodiment the second therapeutic agent is a serotonin-noradrenaline reuptake inhibitor (SNRI) selected from the group consisting of:
Desvenlafaxine (Pristiq)
Duloxetine (Cymbalta, Ariclaim, Xeristar, Yentreve, Duzela)
Milnacipran (Ixel, Savella, Dalcipran, Toledomin)
Venidfaxine (Effexor, Efexor)

In another embodiment the second therapeutic agent is a Noradrenaline reuptake inhibitor (NRI) selected from the group consisting of:
Atomoxetine (Tomoxetine, Strattera, Attentin)
Mazindol (Mazanor, Sanorex)
Reboxetine (Edronax, Norebox, Prolift, Solvex, Davedax, Vestra)
Viloxazine (Vivalan, Emovit, Vivarint, Vicilan)

In another embodiment the second therapeutic agent is a monoamine oxidase inhibitor (MAOI) selected from the group consisting of:
Benmoxin (Nerusil, Neuralex)
Hydralazine (Apresoline)
Iproclozide (Sursum)
Iproniazid (Marsilid, Iprozid, Ipromid, Rivivol, Propilniazida)
Isocarboxazid (Marplan)
Isoniazid (Laniazid, Nydrazid)
Mebanazine (Actomol)
Nialamide (Niamid)
Octamoxin (Ximaol, Nimiaol)
Phenelzine (Nardil, Nardelzine)
Pheniprazine (Catron)
Phenoxypropazine (Drazine)
Pivalylbenzhydrazine (Tersavid)
Procarbazine (Matulane, Natulan, Indicarb)
Caroxazone (Surodil, Timostenil)
Echinopsidine (Adepren)
Furazolidone (Furoxone, Dependal-M)
Linezolid (Zyvox, Zyvoxam, Zyvoxid)
Tranylcypromine (Parnate, Jatrosom)
Brofaromine (Consonar)
Metralindole (Inkazan)
Minaprine (Cantor)
Moclobemide (Aurorix, Manerix)
Pirlindole (Pirazidol)
Toloxatone (Humoryl)
Lazabemide (Pakio, Tempium)
Pargyline (Eutonyl)
Rasagiline (Azilct)
Selegiline (Deprenyl, Eldepryl, Emsam)

In another embodiment the second therapeutic agent is a tricyclic antidepressant (TCA) selected from the group consisting of:
Amitriptyline (Tryptomer, Elavil, Tryptizol, Laroxyl, Sarotex, Lentizol)
Butriptyline (Evadene, Evadyne, Evasidol, Centrolese)
Clomipramine (Anafranil)
Desipramine (Norpramin, Pertofrane)
Dosulepin (Prothiaden, Dothep, Thaden and Dopress)
Doxepin (Aponal, Adapine, Doxal, Deptran, Sinquan, Sinequan. Zonalon, Xepin, Silenor)
Imipramine (Antideprin, Deprimin, Deprinol, Depsol, Depsonil, Dynaprin, Eupramin, Imipramil, Irmin, Janimine, Melipramin, Surplix, Tofranil)
Lofepramine (Gamanil, Tymelyt, Lomont)
Nortriptyline (Sensoval, Aventyl, Pamelor, Norpress, Allegron, Noritren, Nornilen)
Protriptyline (Vivactil)
Trimipramine (Surmontil, Rhotrimine, Stangyl)

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous, and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolon, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propenoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g., inert diluent, preservative disintegrant (e.g., sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The active ingredient can be in micro-encapsulated form with one or more excipients. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g. cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Compositions suitable for topical administration in the mouth include lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol, and water. Transdermal patches may also be used to administer the compounds of the invention.

Compositions for rectal administration may be presented as a suppository with a suitable base comprising, for example, cocoa butter, glycerin, gelatine or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described. An injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In certain embodiments, unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The phrase “pharmaceutically acceptable salt,” as used herein, refers to pharmaceutically acceptable organic or inorganic salts of a provided compound. For use in medicine, the salts of the provided compounds will be pharmaceutically acceptable salts. Other salts may, however, be useful in the preparation of provided compounds or of their pharmaceutically acceptable salts. Pharmaceutically acceptable salts are well known in the art. For example, Berge et al., describe pharmaceutically acceptable salts in detail in J. Pharm. Sci. (1977) 66:1-19, incorporated herein by reference in its entirety. A pharmaceutically acceptable salt involves the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. When multiple charged atoms are present in the parent drug, its pharmaceutically acceptable salts will have multiple counter ions and these can be several instances of the same counter ion or different counter ions. Hence, a pharmaceutically acceptable salt can have one or more charged atoms in the parent compound and/or one or more counter ions.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When a provided compound is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidino, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like. Quarternary ammonium salts such as N⁺(C₁₋₄ alkyl)₄ are also included.

When a provided compound is basic or contains a sufficiently basic bioisostere, salts may be prepared from pharmaceutically acceptable non-toxic acids, including inorganic and organic acids. Such acids include, but are not limited to, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, carbonic, boric, sulfamic, propionic, butyric, hydroxymaleic, mucic, phenylacetic, sulfanilic, aspartic, edetic, stearic, palmitic, oleic, lauric, ascorbic, valeric, perchloric, malonic, p-toluenesulfonic acid and the like. Particular embodiments include citric, hydrobromic, hydrochloric, maleic, phosphoric, sulfuric and tartaric acids. Other exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, palmoate (i.e., 1,1′-methylene-bis-(2-hydroxy-3-naphthoate)), adipate, alginate, ascorbate, aspartate, cyclopentanepropionate, borate, butyrate, camphorate, digluconate, dodecylsulfate, ethanesulfonate, glucoheptonate, glycerophosphate, hemisulfate, heptanoate, hexanoate, 2-hydroxyethanesulfonate, lactobionate, laurate, lauryl sulphate, malonate, 2-naphthalenesulfonate, nicotinate, pectinate, persulfate, 3-phenylpropionate, picrate, pivalate, propionate, stearate, thiocyanate, undecanoate, and valerate salts.

The preparation of the pharmaceutically acceptable salts described above and other typical pharmaceutically acceptable salts is more fully described by Berge et al., “Pharmaceutical Salts,” J. Pharm. Sci., 1977:66:1-19.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for co therapy use, a pharmaceutically acceptable carrier, vehicle or diluent. The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

It will be appreciated that any compound that is a prodrug of a compound of formula (I) or (I′), or a subformula thereof described herein, is also within the scope and spirit of the invention. The term “pro-drug” is used in its broadest sense and encompasses those derivatives that are converted in vivo to the compounds of the invention. Such derivatives would readily occur to those skilled in the art, and include, for example, compounds where a free hydroxy group (for instance at the CR′ position) is converted into an ester, such as an acetate or phosphate ester, or where a free amino group is (for instance at the CR′ position) converted into an amide (e.g., α-aminoacid amide). Procedures for esterifying, e.g., acylating, the compounds of the invention are well known in the art and may include treatment of the compound with an appropriate carboxylic acid, anhydride or chloride in the presence of a suitable catalyst or base.

The compounds of the invention may be in crystalline form either as the free compounds or as solvates (e.g. hydrates) and it is intended that both forms are within the scope of the present invention. Methods of solvation are generally known within the art.

It will also be recognised that compounds of the invention may possess asymmetric centres and are therefore capable of existing in more than one stereoisomeric form. The invention thus also relates to compounds in substantially pure isomeric form at one or more asymmetric centres e.g., greater than about 90% ee, such as about 95% or 97% ee or greater than 99% ee, as well as mixtures, including racemic mixtures, thereof. Such isomers may be prepared by asymmetric synthesis, for example using chiral intermediates, or mixtures may be resolved by conventional methods, e.g. chromatography, or use of a resolving agent.

Furthermore, depending on the substitution pattern the compounds of the present invention may be capable of undergoing tautomerism. Accordingly, all possible tautomers of a compound of the present invention fall within the scope and spirit of the invention.

The synthetic methods and processes described herein to prepare the compounds of the present invention are amenable to solid phase synthetic techniques and/or combinatorial chemistry to produce individual compounds or libraries of compounds.

Traditionally, drug candidates have been synthesised individually, this being a time consuming and laborious process if the synthetic sequence contains even just a few steps and large numbers of compounds are to be evaluated for their biological activity. Combinatorial synthesis is an emerging technique for effecting the generation of large libraries of molecules and has been successfully exploited in the synthesis and evaluation of small organic libraries. These libraries and their starting substrates may exist as molecules in free solution or preferably, linked to a solid support, for example, beads, pins, microtitre plates (wells) or microchips which can be polymeric, glass, silica or other suitable substrate. Chemical diversity can be achieved by either parallel or split (split and mix) syntheses wherein each step has the potential to afford a multitude of compounds.

Solution phase libraries may be prepared via parallel syntheses wherein different compounds are synthesised in separate reaction vessels in parallel, often in an automated fashion. Alternatively, attachment of the individual components employed in a synthetic sequence to an appropriate solid phase support allows for the further creation of chemical diversity by utilising not only parallel synthesis but also split synthesis wherein the solid support containing the compounds prepared in the prior step can be split into a number of batches, treated with the appropriate reagent and recombined.

The substrates can be attached to a solid support surface by any linkers known in the art. The linkers may be any component capable of being cleaved to release the substrate or final compound from the support.

Preferably, the solid support is a polymer support. Examples of polymeric supports currently used in solid phase synthesis include: alkenyl resins: e.g. REM resins; BHA resins: e.g. benzhydrylamine (polymer-bound hydrochloride, 2% crosslinked), benzhydryl chloride (polymer bound); Br-functionalised resins: e.g. brominated PPOA resin, brominated Wang resin; Chloromethyl resins; eg. 4-methoxybenzhydryl chloride (polymer bound); CHO-functionalised resins: eg. indole resin, formylpolystyrene; Cl-functionalised resins: e.g. Merrifield's resin, chloroacetyl (polymer bound); CO₂H-functionalised resins: e.g. carboxypolystyrene; I-functionalised resins: e.g. 4-iodophenol (polymer bound); Janda Jels™; MBHA resins: e.g. 4-methylbenzhydrylamine hydrochloride (polymer bound), 4-hydroxymethylbenzoic acid-4-methyl benzhydrylamine (polymer bound); Amine-functionalised resins: e.g. (aminomethyl)polystyrene, PAL resin, Sieber amide resin; Nitrophenyl carbonate resins: e.g. 4-nitrophenyl carbonate (polymer bound); OH-functionalised resins: e.g. 4-benzyloxybenzyl alcohol (polymer bound); Hydroxy methyl resins: e.g. benzyl alcohol (polymer bound); HMBA resin: Oxime resins; Rink acid resin; Triazine-based resin; Trityl amine resins; Trityl resins: e.g. trityl-chloride (polymer bound), 2-chlorotrityl alcohol, 1,3-diaminepropane trityl.

Thus, individual compounds or libraries of compounds can be synthesised by initially attaching the first compound substrate to a solid support surface which can be performed by providing a plurality of solid support surfaces, suitably derivatising each of the surfaces with groups capable of reacting with either the compound substrate or a linker moiety attached thereto. The various support surfaces with the attached first compound substrate can then be subjected to various reaction conditions and second compound substrates to provide a library of attached compounds, which may, if necessary, be reacted further with third and subsequent compound substrates or varying reactions conditions. Attachment and detachment of substrates and products can be performed under conditions similar to those as described in Johnson, M. O., et al., Tetrahedron, 1999, 55, 11641; Han Y., et al. Tetrahedron 1999, 55, 11669; and Collini, M. D., et al., Tetrahedron Lett., 1997, 58, 7963.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

The entire disclosure of PCT/AU2007/001566, filed Oct. 16, 2007, is incorporated herein by reference.

### EXAMPLES

### Synthetic Protocols

### Example 1

### Preparation of Morpholino 6-(2,3-dihydro-1H-inden-2-yl)amino-1-ethyl-1,4-dihydro-4-oxo-1,8-naphthyridine-3-carboxamide (Example 1h)

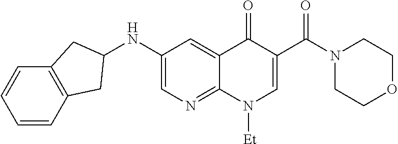 [SMILES: CCn1cc(C(=O)N2CCOCC2)c(=O)c2cc(NC3Cc4ccccc4C3)cnc21]

### Example 1a

### 2-Hydroxy-5-nitronicotinic acid

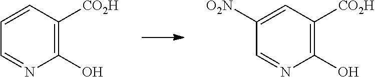 [SMILES: O=C(O)c1cccnc1O ; O=C(O)c1cc([N+](=O)[O-])cnc1O]

To 2-hydroxynicotinic acid (3.6 mmol) in sulfuric acid (30% free SO₃, 2 ml) was added sodium nitrate (7.2 mmol) portionwise over 20 min. The solution was allowed to stir for 20 h at room temperature. The solution was then poured onto ice-water and the precipitate that formed was filtered off, washed with water and dried in a vacuum oven to afford a pale yellow solid (45%).

ESIMS: M-1: 183

¹H NMR (300 MHz, DMSO) δ 8.94 (1H, d, H-4), 8.67 (1H, d, H-6).

### Example 1b

### 2-Chloro-5-nitronicotinic acid

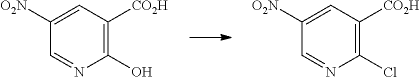 [SMILES: O=C(O)c1cc([N+](=O)[O-])cnc1O ; O=C(O)c1cc([N+](=O)[O-])cnc1Cl]

2-Hydroxy-5-nitronicotinic acid (2.7 mmol) in a mixture of N,N-dimethylformamide (2.7 mmol) and thionyl chloride (5 ml) was heated at 80° C. for 1 h. The mixture was allowed to cool and concentrated in vacuo. To the resulting residue was added ice-water (20 ml) and with vigorous stirring a precipitate formed. The precipitate was filtered off and dried in a vacuum oven to give a white solid (68%).

ESIMS: M-1: 201.

¹H NMR (300 MHz, DMSO) δ 9.30 (1H, d, H-4), 8.83 (1H, d, H-6).

### Example 1c

### 2-Methoxy-5-nitro-nicotinic acid

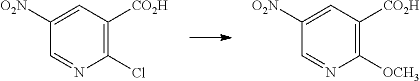 [SMILES: O=C(O)c1cc([N+](=O)[O-])cnc1Cl ; COc1ncc([N+](=O)[O-])cc1C(=O)O]

To 2-chloro-5-nitronicotinic acid (1.0 mmol) in methanol was added a solution of sodium methoxide in methanol (2.4 mmol, freshly prepared from sodium metal in methanol). The solution was refluxed for 2 h and the mixture was allowed to cool and concentrated in vacuo. To the resulting residue was added 10% citric acid solution (20 ml) and the solution extracted with ethyl acetate (20 ml). The organic layer was dried (MgSO₄) and concentrated in vacuo. The residue was crystallised from water to give a yellow-white solid (73%).

ESIMS: M-1 197.

¹H NMR (300 MHz, DMSO) δ 9.30 (1H, d, H-4), 8.83 (1H, d, H-6), 4.05 (3H, s, OCH₃).

### Example 1d

### Ethyl 3-oxo-3-(5-nitro-2-methoxypyridin-3-yl)propionate

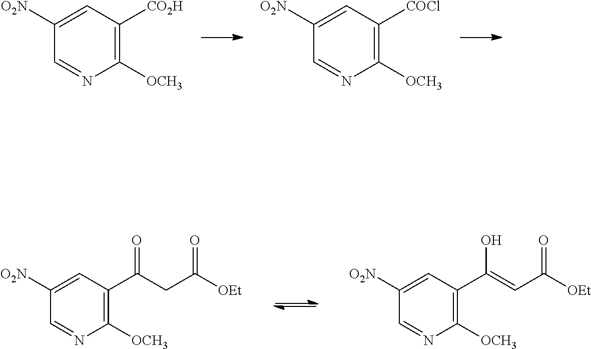 [SMILES: COc1ncc([N+](=O)[O-])cc1C(=O)O ; COc1ncc([N+](=O)[O-])cc1C(=O)Cl ; CCOC(=O)CC(=O)c1cc([N+](=O)[O-])cnc1OC ; CCOC(=O)/C=C(\O)c1cc([N+](=O)[O-])cnc1OC]

2-Methoxy-5-nitronicotinic acid (36 mmol) and phosphorous pentachloride (72 mmol) were heated at 100° C. for 2 h. The excess reagent was removed in vacuo to give an oily residue.

To a solution of ethyl potassium malonate (75.6 mmol) and triethylamine (72 mmol) in acetonitrile (110 ml) was added magnesium chloride (90 mmol) portionwise over 10 min. This solution was allowed to stir for 8 h at 35° C. To this solution was added dropwise a solution of the pyridyl chloride (from above) in acetonitrile (15 ml) at 0° C. over 20 min. The solution was allowed to warm to room temperature and stirred for 20 h. To this solution was added diethyl ether (100 ml) and 1N hydrochloric acid solution until the pH 5-6 was reached. The two layers were separated and the organic layer was washed with water (100 ml). The organic layer was then dried (MgSO₄) and concentrated in vacuo. The resulting residue was then subjected to column chromatography eluting with dichloromethane to afford a clear oily liquid (78%). The NMR spectrum of this compound showed evidence of ketone-enol tautomerism.

ESIMS: M-1: 267.

¹H NMR (300 MHz, DMSO) δ 9.17 (d, 0.6H), 9.05 (d, 0.4H), 8.96 (d, 0.4H), 8.94 (d, 0.6H), 6.20 (s, 0.4H), 4.31-4.13 (m, 5H, OMe+OCH₂), 3.99 (s, 1.2H), 1.33 (t, 3×0.4H), 1.22 (t, 3×0.6H).

### Example 1e

### Ethyl 1-ethyl-1,4-dihydro-6-nitro-4-oxo-1,8-naphthyridine-3-carboxylate

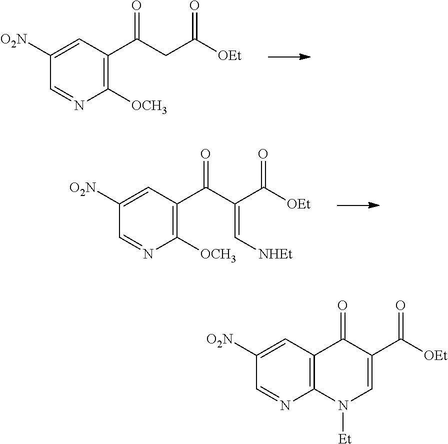

The pyridyl malonate (18 mmol) and triethylorthoformate (23.4 mmol) in acetic anhydride (8 ml) were refluxed for 1 h. The solution was allowed to cool and the excess acetic anhydride was distilled off in vacuo. To the resulting residue in acetonitrile (40 ml) was added dropwise ethylamine (36 mmol) in diethyl ether (20 ml) and the solution was allowed to stir for 5 h at room temperature. The solution was then allowed to cool and was concentrated in vacuo. The residue was dissolved in dichloromethane (60 ml) and washed with water (2×60 ml). The organic layer was then dried (MgSO₄) and concentrated in vacuo. The resulting residue was subjected to column chromatography eluting with 100% dichloromethane, and then 2% methanol/dichloromethane to give a white solid (78%).

ESIMS: M+1: 292.

¹H NMR (300 MHz, CDCl₃) δ 9.50 (1H, d, H-5), 9.44 (1H, d, H-7), 8.66 (1H, s, H-2), 4.53 (2H, q, OCH₂), 4.39 (2H, q, NCH₂), 1.51 (3H, t, OCH₂CH₃), 1.40 (3H, t, NCH₂CH₃).

### Example 1f

### Ethyl 1-ethyl-1,4-dihydro-6-amino-4-oxo-1,8-naphthyridine-3-carboxylate

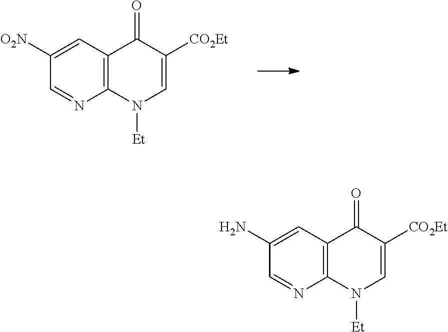 [SMILES: CCOC(=O)c1cn(CC)c2ncc([N+](=O)[O-])cc2c1=O ; CCOC(=O)c1cn(CC)c2ncc(N)cc2c1=O]

The naphthyridine (1.7 mmol) in N,N-dimethyl formamide (10 ml) was hydrogenated over Raney nickel (0.17 mmol) for 4 h at rt. The mixture was filtered through Celite and washed with tetrahydrofuran. The filtrate was evaporated to dryness. Crystallisation from ethanol obtained the residue as a pale yellow solid (67%).

ESIMS: M+1: 262.

¹H NMR (300 MHz, DMSO) δ 8.65 (1H, s, ArH), 8.22 (1H, d, J=2.9 Hz, ArH), 7.62 (1H, d, J=2.9 Hz, ArH), 5.76 (2H, s, NH₂), 4.39 (2H, q, J=6.9 Hz, OCH₂), 4.17 (2H, q, J=7.2 Hz, NCH₂), 1.24 (3H, t, J=6.9 Hz, OCH₂CH), 1.23 (3H, t, J=7.0 Hz, NCH₂CH₃).

### Example 1g

### Ethyl 6-(2,3-dihydro-1H-inden-2-ylamino)-1-ethyl-1,4-dihydro-4-oxo-1,8-naphthyridine-3-carboxylate

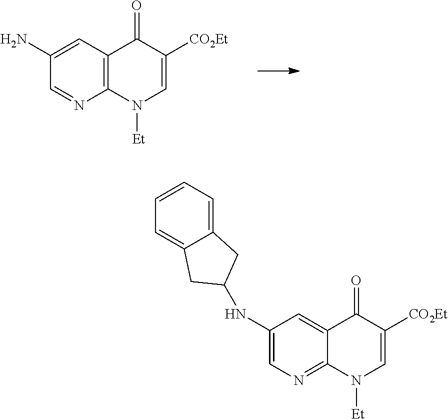 [SMILES: CCOC(=O)c1cn(CC)c2ncc(N)cc2c1=O ; CCOC(=O)c1cn(CC)c2ncc(NC3Cc4ccccc4C3)cc2c1=O]

A stirred solution of the 6-aminonaphthyridinone above (0.1 mmol), sodium sulfate (1.0 mmol), 2-indanone (0.15 mmol) and AcOH (7.5 ml) in dichloroethane (30 ml) under a nitrogen atmosphere was allowed to mature for 15 mins at room temperature. Sodium triacetoxyborohydride (0.15 mmol) was then added in one portion and the solution was allowed to stir for 4 h at rt (the reaction was monitored by TLC). A second addition of sodium sulfate (1.0 mmol), 2-indanone (0.15 mmol) and sodium triacetoxyborohydride (0.15 mmol) and stirring overnight was required to drive the reaction to completion. The reaction mixture was quenched with 10% sodium hydrogen carbonate solution and dichloromethane added to dilute the solution. The organic layer was separated from the aqueous layer and the organic layer dried (MgSO₄). The organic layer was concentrated in vacuo and the resulting residue subjected to silica column chromatography, gradient-eluting with 100% dichloromethane and then 1% MeOH/dichloromethane to give an oily residue. The residue was triturated using diethyl ether and the solid was filtered off at the pump to afford a pale yellow solid (78%).

ESIMS: M+1: 378.

¹H NMR (300 MEz, CDCl₃) δ 8.64 (1H, s, H-2), 8.30 (1H, d, H-5), 7.55 (1H, d, H-7), 7.23-7.10 (4H, m, 4×ArH), 6.69 (1H, d, NH), 4.41 (2H, q, OCH₂), 4.38-4.23 (1H, m, NCH), 4.17 (2H, q, NCH₂), 3.32 (2H, dd, CHCH₂), 2.81 (2H, dd, CHCH₂), 1.32 (3H, t, OCH₂CH₃), 1.25 (3H, t, NCH₂CH₃).

### Example 1h

### Morpholino 6-(2,3-dihydro-1H-inden-2-ylamino)-1-ethyl-1,4-dihydro-4-oxo-1,8-naphthyridine-3-carboxamide

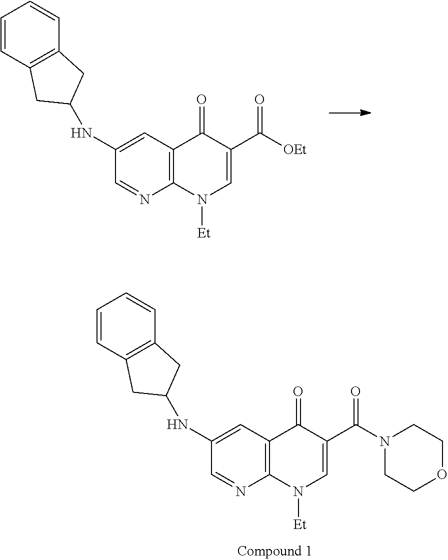 [SMILES: CCOC(=O)c1cn(CC)c2ncc(NC3Cc4ccccc4C3)cc2c1=O ; CCn1cc(C(=O)N2CCOCC2)c(=O)c2cc(NC3Cc4ccccc4C3)cnc21]

Trimethylaluminium (0.8 mmol, 2M in toluene) was added dropwise to a stirred solution of morpholine (0.8 mmol) in dichloromethane (5 ml). The mixture was stirred for 15 mins and then the naphthyridine (0.4 mmol) in dichloromethane (5 ml) was added. The mixture was then stirred for 20 h at 35° C. The mixture was cooled and then quenched by adding 2 N hydrochloric acid (10 ml) dropwise. The organic layer was then separated, dried (MgSO₄) and concentrated in vacuo. The resulting residue was triturated with diethyl ether to give a white solid (78%).

ESIMS: M+1: 419.

¹H NMR (300 MHz, CDCl₃) δ 8.16 (1H, d, ArH), 8.04 (1H, s, ArH), 7.76 (d, 1H, ArH), 7.24-7.12 (m, 4H, ArH), 4.47-4.33 (4H, m-complex, NH—CH+CH₂CH₃), 3.75 (s, 6H, morpholino), 3.42-3.35 (m-complex, 4H, morpholino+CHCH₂), 2.87 (dd, 2H, CHCH₂), 1.43 (t, 3H, CH₃).

### Example 2

### Preparation of Methylpiperazino 6-(2,3-dihydro-1H-inden-2-ylamino)-1-ethyl-1,4-dihydro-4-oxo-1,8-naphthyridine-3-carboxamide (Example 2)

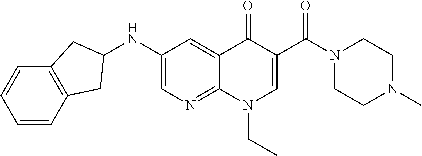 [SMILES: CCn1cc(C(=O)N2CCN(C)CC2)c(=O)c2cc(NC3Cc4ccccc4C3)cnc21]

Trimethylaluminium (1 ml, 2M in toluene) was injected via syringe into a stirred solution of 1-methylpiperazine (100 mg, 1 mmol) in DCM (10 ml). The reaction was stirred at room temperature for 1 h and then treated with ethyl 6-(2,3-dihydro-1H-inden-2-ylamino)-1,4-dihydro-4-oxo-1,5-naphthyridine-3-carboxylate (188.5 mg, 0.5 mmol). The resulting mixture was stirred at room temperature for 16 h and then poured into 5 ml of 2M HC aq. The organic compound was extracted with ethyl acetate (3×10 ml) and the combined extract was dried over MgSO₄, filtered and concentrated under reduced pressure to afford the crude oil (120 mg crude). Chromatography of a small quantity of the crude gave the desired product (40 mg).

ESIMS: m/z=432.0 [M+H]⁺.

¹H-NMR (300 MHz, CDCl₃): δ 8.15 (d J=2.2, 1H), 8.09 (s, 1H), 7.80 (d, J=2.2 Hz, 1H), 7.15-7.25 (m, 4H), 4.30-4.50 (m, 4H), 3.78 (s, 2H), 3.35-3.50 (m, 4H), 2.86 (dd, 1.1 Hz, 2H), 2.48 (t, J=2 Hz, 4H), 2.30 (s, 3H), 1.47 (, J=2.4 Hz, 3H).

### Example 3

### Preparation of Cyclopropylamino 6-(2,3-dihydro-1H-inden-2-ylamino)-1-ethyl-1,4-dihydro-4-oxo-1,8-naphthyridine-3-carboxamide (Example 3)

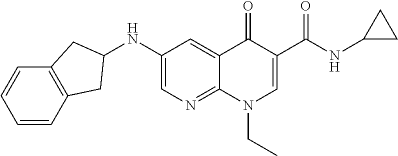 [SMILES: CCn1cc(C(=O)NC2CC2)c(=O)c2cc(NC3Cc4ccccc4C3)cnc21]

Trimethylaluminium (1 ml, 2M in toluene) was injected via syringe into a stirred solution of cyclopropylamine (57 mg, 1 mmol) in DCM (10 ml). The reaction was stirred at room temperature for 1 h and then treated with ethyl 6-(2,3-dihydro-1H-inden-2-ylamino)-1,4-dihydro-4-oxo-1,5-naphthyridine-3-carboxylate (188.5 mg, 0.5 mmol). The resulting mixture was stirred at room temperature for 16 h and then poured into 5 ml of 2M HCl aq. solution. The organic compound was extracted with ethyl acetate (3×10 ml) and the combined extract was dried over MgSO₄, filtered and concentrated under reduced pressure to afford the crude oil (150 mg crude). Chromatography of a small quantity of the crude gave the desired product (80 mg).

ESIMS: m/z=389.0 [M+H]⁺.

¹H-NMR (300 MHz, CDCl₃): δ 10.01 (s, 1H), 8.81 (s, 1H), 8.18 (s, 1H), 7.80 (s, 1H), 7.15-7.25 (m, 4H), 430-4.51 (m, 4H), 3.42 (dd, J=5.0, 5.0 Hz, 2H), 2.88-2.97 (m, 3H), 1.45 (t, J=4.2 Hz, 3H); 0.77-0.82 (m, 2H), 0.60-0.65 (m, 2H).

### Example 4

### Preparation of Morpholino 6-amino-1-ethyl-1,4-dihydro-4-oxo-1,8-naphthyridine-3-carboxamide (Example 4b)

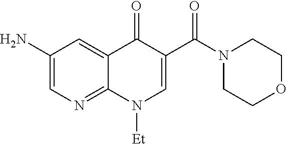 [SMILES: CCn1cc(C(=O)N2CCOCC2)c(=O)c2cc(N)cnc21]

### Example 4a

### Morpholino 6-amino-1-ethyl-1,4-dihydro-4-oxo-1,8-naphthyridine-3-carboxamide

Trimethylaluminium (2.0 mmol, 2M in toluene) was added dropwise to a stirred solution of morpholine (2.0 mmol) in dichloromethane (20 ml). The mixture was stirred for 15 mins and then ethyl 1-ethyl-1,4-dihydro-6-nitro-4-oxo-1,8-naphthyridine-3-carboxylate (1.0 mmol) in dichloromethane (20 ml) was added. The mixture was then stirred for 7 d at 40° C. The mixture was cooled and then quenched by adding 2 N hydrochloric acid dropwise. The organic layer was then separated, dried (MgSO₄) and concentrated in vacuo. The resulting residue was triturated with diethyl ether to give morpholino 6-amino-1-ethyl-1,4-dihydro-4-oxo-1,8-naphthyridine-3-carboxamide, which was used directly in the next step.

ESIMS: m/z=332.90 [M+H]⁺.

### Example 4b

### Morpholino 6-amino-1-ethyl-1,4-dihydro-4-oxo-1,8-naphthyridine-3-carboxamide

Morpholino 1-ethyl-1,4-dihydro-6-nitro-4-oxo-1,8-naphthyridine-3-carboxamide (0.06 mmol) in N,N-dimethyl formamide (1 ml) and ethanol (5 mL) was hydrogenated over Raney nickel (0.09 mmol) for 2 h at rt. The mixture was filtered through Celite and washed with tetrahydrofuran. The filtrate was evaporated to dryness to give morpholino 6-amino-1-ethyl-1,4-dihydro-4-oxo-1,8-naphthyridine-3-carboxamide.

ESIMS: m/z=303.0 [M+H]⁺.

¹H-NMR (300 MHz, CDCl₃): δ 8.25 (d, J=2.3, 1H), 8.07 (s, 1H), 7.90 (s, J=2.3 Hz, 1H), 4.39 (q, J=7.0 Hz, 2H), 4.04 (m, 2H), 3.76 (m, 6H), 3.42 (m, 2H), 1.46 (t, J=7.0 Hz, 3H).

### Example 5

### Preparation of 4-fluorophenylamine 6-(2,3-dihydro-1H-inden-2-ylamino)-1-ethyl-1,4-dihydro-4-oxo-1,8-naphthyridine-3-carboxamide (Example 5)

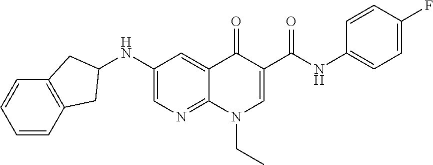 [SMILES: CCn1cc(C(=O)Nc2ccc(F)cc2)c(=O)c2cc(NC3Cc4ccccc4C3)cnc21]

Trimethylaluminium (1 ml, 2M in toluene) was injected via syringe into a stirred solution of 4-fluoroaniline (111 mg, 1 mmol) in DCM (10 ml). The reaction was stirred at room temperature for 1 h and then treated with ethyl 6-(2,3-dihydro-1H-inden-2-ylamino)-1,4-dihydro-4-oxo-1,5-naphthyridine-3-carboxylate (188.5 mg, 0.5 mmol). The resulting mixture was stirred at room temperature for 16 h and then poured into 5 ml of 2M HCl aq. Solution. The organic compound was extracted with ethyl acetate (3×10 ml) and the combined extract was dried over MgSO₄, filtered and concentrated under reduced pressure to afford the crude (160 mg). Chromatography of a small quantity of the crude gave the desired product (90 mg).

ESIMS: m/z=443.0 [M+H]⁺.

¹H-NMR (300 MHz, CDCl₃): δ 8.86 (s, 1H), 8.20 (d. J=1.8 Hz, 1H), 7.83 (d, J=1.8 Hz, 1H), 7.69-7.74 (m, 2H), 7.16-7.24 (m, 4H), 6.98-7.04 (t, J=7 Hz, 2H), 4.50 (q, J=14, 6 Hz, 2H), 4.44 (s broad, 2H), 3.45 (d, J=14 Hz, 2H), 2.91 (d, J=14 Hz, 2H), 1.46 (t, J=8 Hz, 3H).

### Example 6

### Preparation of 4-biphenylamino-6-(2,3-dihydro-1H-inden-2-ylamino)-1-ethyl-1,4-dihydro-4-oxo-1,8-naphthyridine-3-carboxamide (Example 6)

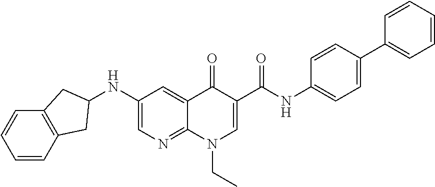 [SMILES: CCn1cc(C(=O)Nc2ccc(-c3ccccc3)cc2)c(=O)c2cc(NC3Cc4ccccc4C3)cnc21]

Trimethylaluminium (1 ml, 2M in toluene) was injected via syringe into a stirred solution of 4-aminobiphenyl (169 mg, 1 mmol) in DCM (10 ml). The reaction was stirred at room temperature for 1 h and then treated with ethyl 6-(2,3-dihydro-1H-inden-2-ylamino)-1,4-dihydro-4-oxo-1,5-naphthyridine-3-carboxylate (188.5 mg, 0.5 mmol). The resulting mixture was stirred at room temperature for 16 h and then poured into 5 ml of 2M HCl aq. solution. The organic compound was extracted with ethyl acetate (3×10 ml) and the combined extract was dried over MgSO₄, filtered and concentrated under reduced pressure to afford the crude product (140 mg). Chromatography of a small quantity of the crude gave the desired product (50 mg).

ESIMS: m/z=501.0 [M+H]⁺.

¹H-NMR (300 MHz, CDCl₃): δ 8.90 (s, 1H), 8.21 (d, J=3.5 Hz, 1H), 7.84-7.88 (m, 3H), 7.57-7.61 (m, 4H), 7.41 (t, J=6.8 Hz, 3H), 7.16-7.32 (m, 5H), 4.52 (q, J=10, 6 Hz, 2H), 4.37-4.44 (m, 2H), 3.46 (dd, J=15, 5 Hz, 2H), 2.92 (dd, J=15, 3 Hz, 2H), 1.52 (t, J=6 Hz, 3H).

### Example 7

### Preparation of Ethyl 6-(isobutyrylamide)-1-ethyl-1,4-dihydro-4-oxo-1,8-naphthyridine-3-carboxylate (Example 7)

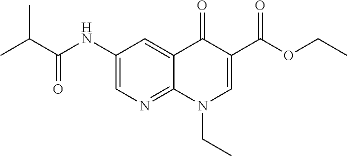 [SMILES: CCOC(=O)c1cn(CC)c2ncc(NC(=O)C(C)C)cc2c1=O]

To the solution of ethyl 1-ethyl-1,4-dihydro-6-imino-4-oxo-1,8-naphthalidine-3-carboxylate (261 mg, 1 mmol) in dichloromethane (5 ml) was treated with isobutyryl chloride (213 mg, 2 mmol) at 5° C. The reaction mixture was then heated to 60° C. for 16 h. The reaction mixture was poured into ice and the product was extracted with ethyl acetate (3×10 ml). The combined extract was dried over MgSO₄ and concentrated under reduced pressure to afford the crude product (220 mg). Chromatography of a small quantity of the crude using 10% ethyl acetate in hexane afforded the desired product (NMR data indicates that the product was a mixture of cis and trans-isomers (due to amide linkage).

ESIMS: m/z=332.0 [M+H]⁺.

¹H-NMR (300 MHz, CDCl₃): δ 9.74 (s), 9.54 (d, J=2.5 Hz.), 9.3-9.31 (m), 8.85 (s), 8.72 (d, J=2.5 Hz.), 8.66 (s), 8.62 (d, J=2.5 Hz.), 8.59 (s), 4.43-4.53 (m, 2H), 428-4.35 (m, 2H), 3.41-3.50 (m, 2H), 2.77-2.86 (m, 1H), 2.60-2.69 (m, 1H), 1.41-1.50 (m), 1.31-1.36 (t), 1.18-1.25 (m).

### Example 8

### Preparation of Morpholino 6-(2,3-dihydro-1H-inden-2-ylamino)-1-ethyl-1,4-dihydro-4-oxo-1,5-naphthyridine-3-carboxamide (Example 8)

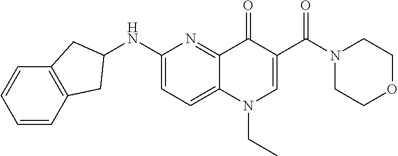 [SMILES: CCn1cc(C(=O)N2CCOCC2)c(=O)c2nc(NC3Cc4ccccc4C3)ccc21]

### a) N-(2,3-Dihydro-1H-inden-2-yl)-5-nitropyridin-2-amine

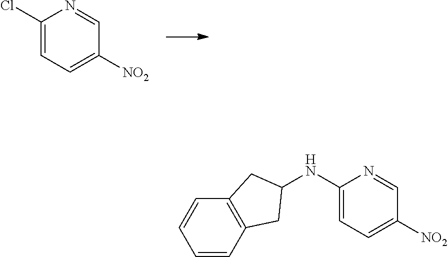 [SMILES: O=[N+]([O-])c1ccc(Cl)nc1 ; O=[N+]([O-])c1ccc(NC2Cc3ccccc3C2)nc1]

A mixture of 2-chloro-5-nitropyridine (4 g) and N,N-diisopropylethylamine (3 ml) was heated under reflux in dry ethanol (100 ml) for 2 h. The reaction mixture was cooled to 0° C., the solid which separated was filtered off washed with little cold ethanol, dried to give the product (6.25 g, 88%).

¹H NMR (300 MHz, CDCl₃) δ 8.98 (1H, s, H-6), 8.23 (1H, d, H-4), 7.26-7.17 (4H, m, Ar), 6.43 (1H, d, H-3), 6.12 (1H, bs, NH), 4.71 (1H, bs, NCH), 3.44 (2H, dd, CHCH₂), 2.93 (2H, dd CHCH₂).

ESIMS: M+1: 256.

### b) N²-(2,3-Dihydro-1H-inden-2-yl)pyridine-2,5-diamine & Diethyl 2-((6-(2,3-dihydro-1H-inden-2-ylamino)pyridine-3-ylamino)methylene)malonate

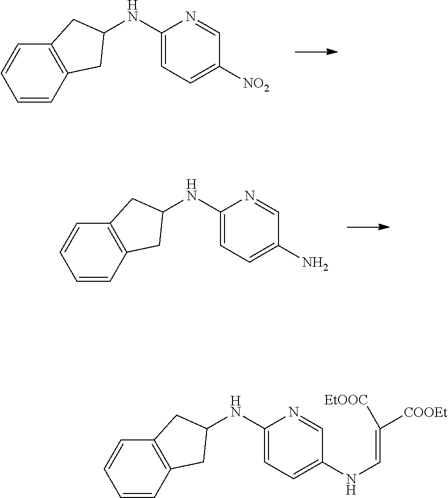 [SMILES: O=[N+]([O-])c1ccc(NC2Cc3ccccc3C2)nc1 ; Nc1ccc(NC2Cc3ccccc3C2)nc1 ; CCOC(=O)C(=CNc1ccc(NC2Cc3ccccc3C2)nc1)C(=O)OCC]

A mixture of N-(2,3-dihydro-1H-inden-2-yl)-5-nitropyridin-2-amine (5.5 g) and Rancy Nickel (50 mg) was stirred in DMF (30 ml) under hydrogen overnight. The reaction mixture was filtered through celite and the solvent removed in vacuo, giving as a residue crude N²-(2,3-dihydro-1H-inden-2-yl)pyridine-2,5-diamine, which was on-reacted without further characterization other than ascertaining that the compound was one spot by tlc with the expected molecular weight (M+1) of 226.

A crude mixture of N²-(2,3-dihydro-1H-inden-2-yl)pyridine-2,5-diamine (5 g) and diethyl ethoxymethylenemalonate (5.5 g) was heated under reflux in dry diethyl ether (50 ml) for 1 h. The reaction mixture was then cooled to room temperature, and solvent removed under reduced pressure and the remaining residue finally recrystallised from acetonitrile to give the diethyl 2-((6-(2,3-dihydro-1H-inden-2-ylamino)pyridin-3-ylamino)methylene)malonate (6 g).

¹H NMR (300 MHz, DMSO-d₆) δ 10.58 (1H, d, 2-NHCH), 8.15 (1H, d, 5-NHCH), 8.02 (1H, s, Ar), 7.45 (1H, dd, Ar), 7.20-7.10 (4H, m, Ar), 6.49 (1H, d, 2-NH), 4.51 (1H, m, 2-NHCH), 6.16-4.04 (4H, 2q, 2×OCH₂), 3.44 (2H, dd, CHCH₂), 2.93 (2H, dd CHCH₂), 1.24-1.16 (6H, 2t, OCH₂CH₃).

ESIMS: M+1: 396.

### c) Ethyl 6-(2,3-dihydro-1H-inden-2-yl)amino-1,4-dihydro-4-oxo-1,5-naphthyridine-3-carboxylate

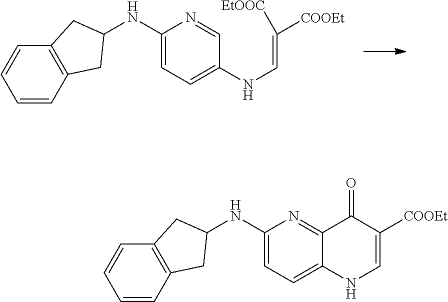 [SMILES: CCOC(=O)C(=CNc1ccc(NC2Cc3ccccc3C2)nc1)C(=O)OCC ; CCOC(=O)c1c[nH]c2ccc(NC3Cc4ccccc4C3)nc2c1=O]

A solution of diethyl 2-((6-(2,3-dihydro-1H-inden-2-yl)amino-pyridin-3-ylamino)methylene)malonate (1.3 g) in dichloromethane (10 ml) was added very carefully into pre-heated (230° C.) diphenyl ether (20 ml) with stirring and heating was continued with stirring for another 20 min after addition had been completed. This was then allowed to cool to room temperature, petroleum ether (200 ml) was added, and the solid which separated was filtered off dried to give the crude product (600 mg), which was on-reacted without further characterization other than ascertaining that the compound was one spot by tlc with the expected molecular weight (M+1) of 350.

### d) Ethyl 6-(2,3-dihydro-1H-inden-2-ylamino)-1-ethyl-1,4-dihydro-4-oxo-1,5-naphthyridine-3-carboxylate

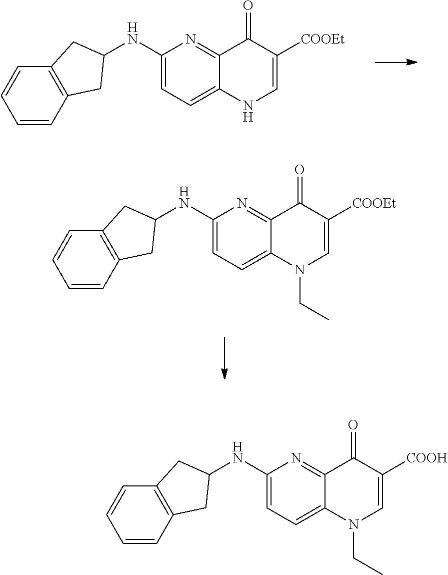 [SMILES: CCOC(=O)c1c[nH]c2ccc(NC3Cc4ccccc4C3)nc2c1=O ; CCOC(=O)c1cn(CC)c2ccc(NC3Cc4ccccc4C3)nc2c1=O ; CCn1cc(C(=O)O)c(=O)c2nc(NC3Cc4ccccc4C3)ccc21]

A mixture of ethyl 6-(2,3-dihydro-1H-inden-2-ylamino)-1,4-dihydro-4-oxo-1,5-naphthyridine-3-carboxylate (600 mg), iodoethane (1 g) and potassium carbonate (600 mg) were heated at 90° C. in DMF (20 ml) overnight. After a standard ethyl acetate/aqueous work-up, the residue from the evaporated organic layer (crude cyclized ethyl ester), was heated at 80° C. in a mixture of ethanol (25 ml) and 2M NaOH (10 ml) for 2 h. This was then cooled to room temperature, aq. HCl was added to adjust the pH to 5, at which point the product precipitated and was filtered off, washed with water, and dried to give the crude product (˜350 mg).

¹H NMR (300 MHz, DMSO-d₆) δ 8.78 (1H, s, H-2), 8.10 (1H, dd, H-8), 7.71 (1H, d, H-7), 7.24-7.11 (4H, m, Ar), 7.06 (1H, d, NH), 4.75 (1H, m, NHCH), 4.46 (2H, q, NCH₂), 3.32 (2H, dd, CHCH₂), 2.83 (2H, dd CHCH₂), 1.33 (3H, t, NCH₂CH₃); and ESIMS: M+1: 350.

### e) Morpholino 6-(2,3-dihydro-1H-inden-2-yl)amine-1-ethyl-1,4-dihydro-4-oxo-1,5-naphthyridine-3-carboxamide

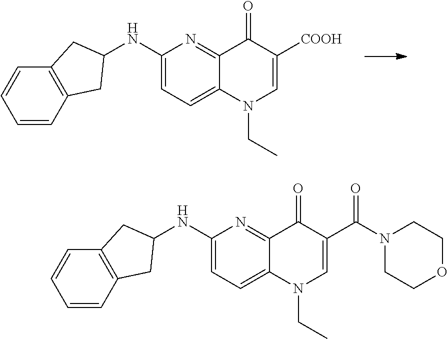

A mixture of 6-(2,3-dihydro-1H-inden-2-yl)amino-1-ethyl-1,4-dihydro-4-oxo-1,5-naphthyridine-3-carboxylic acid (200 mg), HBTU (250 mg) and N,N-diisopropylethylamine (150 mg) in dry DMF (1.5 ml) were stirred for 1 h at RT. Finally morpholine (100 mg) was added and the reaction mixture was stirred overnight. After a standard ethyl acetate/aqueous work-up, the residue from the evaporated organic layer was purified by passing through silica gel column using acetone as a mobile phase to give the 6-(2,3-dihydro-1H-inden-2-ylamino)-1-ethyl-1,4-dihydro-oxo-4-oxo-1,5-naphthyridine-3-(˜150 mg).

¹H NMR (300 MHz, DMSO-d₆) δ 8.00 (1H, s, H-2), 7.89 (1H, dd. H-8), 7.30 (1H, d, H-7), 7.22-7.10 (4H, m, Ar), 6.93 (1H, d, NH), 4.75 (1H, m, NHCH), 4.23 (2H, q, NCH₂), 3.56-3.27 (8H, bm, morpholino), 3.27 (2H, dd, CHCH₂), 2.76 (2H, dd CHCH₂), 1.27 (3H, t, NCH₂CH₃); and

ESIMS: M+1: 419.

### Example 9

### Preparation of morpholino 6-(2,3-dihydro-1H-inden-2-ylamino)-1-ethyl-1,4-dihydro-4-oxo-1,7-naphthyridine-3-carboxamide (Example 9)

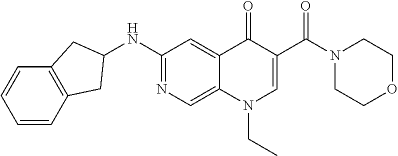 [SMILES: CCn1cc(C(=O)N2CCOCC2)c(=O)c2cc(NC3Cc4ccccc4C3)ncc21]

### a) 2,5-Dichloropyridine-4-carboxylic acid

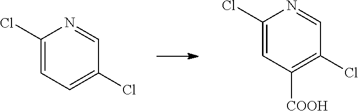 [SMILES: Clc1ccc(Cl)nc1 ; O=C(O)c1cc(Cl)ncc1Cl]

At −75° C., 2,5-dichloropyridine (3.7 g) was added to a solution of butyl lithium (25 ml, 1M) and N,N,N′,N″,N″-pentamethyldiethylenetriamine (5.3 ml) in THF (50 ml) under a nitrogen atmosphere at −75° C. and the reaction mixture stirred for 2 h, poured onto dry ice, and water (50 ml) added. The aqueous phase was washed with diethyl ether; acidified to pH 2 and the white solid filtered off dried to give the product (2.5 g), a known compound, for the next reaction, without further characterization other than ascertaining that the compound was one spot by tlc with the expected molecular weight (M−1) of 190.

### b) Ethyl 3-(2,5-dichloropyridin-4-yl)-3-oxopropionoate

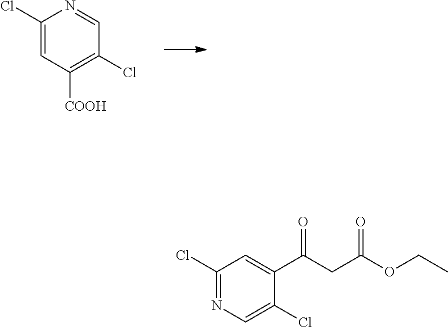 [SMILES: O=C(O)c1cc(Cl)ncc1Cl ; CCOC(=O)CC(=O)c1cc(Cl)ncc1Cl]

A mixture of 2,5-dichloropyridine-4-carboxylic acid (2 g) and SOCl₂ (10 ml) and 1 drop of DMF were heated under reflux for 2 h, and all SOCl₂ and DMF removed under reduced pressure to give the crude acid chloride as the remaining residue. Separately, a suspension of potassium ethyl malonate (5 g) in acetonitrile (100 ml) was cooled to 0° C., magnesium chloride (4 g) and triethylamine (4 ml) were added, the ice bath removed and the reaction stirred at RT for 3 h. A solution of the crude acid chloride in DCM (25 ml) was carefully added to the malonate slurry and the resulting mixture stirred at RT overnight. Aqueous HCl (100 ml, 1M) was added and stirring continued for 1 h. This mixture was then extracted with diethyl ether (200 ml×3), the organic layer washed with saturated sodium bicarbonate (200 ml×2) and brine (200 ml), dried over anhydrous magnesium sulfate, filtered and concentrated. The title compound was obtained as light yellow oil (1.6 g) which was on-reacted without further purification and without further characterization other than ascertaining that the compound was one spot by tlc with the expected molecular weight (M−1) of 260.

### c) Preparation of 3-(2,5-dichloropyridin-4-yl)-2-(2-ethylamino)-ethylene-1-yl)-3-oxopropanoate

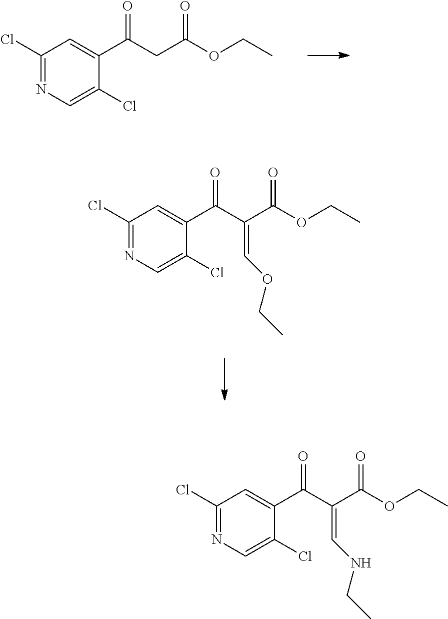 [SMILES: CCOC(=O)CC(=O)c1cc(Cl)ncc1Cl ; CCO/C=C(\C(=O)OCC)C(=O)c1cc(Cl)ncc1Cl ; CCN/C=C(\C(=O)OCC)C(=O)c1cc(Cl)ncc1Cl]

A solution of ethyl 3-(2,5-dichloropyridin-4-yl)-3-oxopropanoate (1.6 g) and triethylorthoformate (1.6 mL) in acetic anhydride (6 ml) was heated at 130° C. for 2 h with stirring. After cooling to RT, all solvent was removed in vacuo, toluene added, removed in vacuo, and this procedure repeated once more. The remaining crude residue was re-dissolved in THF (50 mL) and the ethyl amine (70% in water, 5 ml) was added dropwise with stirring at RT and stirring continued further for 3 h. The reaction mixture was than extracted with DCM (200 ml×3), the organic layer washed with water, dried over magnesium sulfate, filtered, and then all DCM removed under reduced pressure to give the crude product. This crude product was triturated with diethyl ether to give the pure Example 3c as a mixture of E,Z-isomers of the enamine (1.5 g).

ESIMS: m/z 317.0 [M+H]⁺.

¹H NMR (300 MHz, CDCl₃): δ 11.05 (bs, 0.8H, NH), 9.75 (bs, 0.2H, NH), 8.34 (s, 1H), 8.24 (s, 0.5H), 8.19 (s, 0.5H), 7.15 (s, 1H), 3.9-4.1 (m, 2H), 3.5-3.6 (m, 2H), 1.41 (t, 3H), 1.03 (t, 3H).

### d) Preparation of Ethyl 6-chloro-1-ethyl-1,4-dihydro-4-oxo-1,7-naphthyridine-3-carboxylate

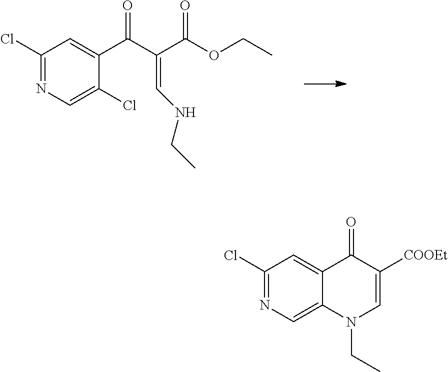 [SMILES: CCN/C=C(\C(=O)OCC)C(=O)c1cc(Cl)ncc1Cl ; CCOC(=O)c1cn(CC)c2cnc(Cl)cc2c1=O]

A mixture of 3-(2,5-dichloropyridin-4-yl)-2-(2-ethylamino)-ethylene-1-yl)-3-oxopropanoate (1.2 g) and potassium carbonate (1 g) was heated at 100° C. in DMF (30 ml) for 12 hours. After a standard ethyl acetate/aqueous work-up, the residue from the evaporated organic layer gave the product, ethyl 6-chloro-1-ethyl-1,4-dihydro-4-oxo-1,7-naphthyridine-3-carboxylate (1 g).

ESIMS: m/z 281.0 [M+H]⁺.

¹H NMR (300 MHz, CDCl₃): δ 8.80 (s, 1H), 8.53 (s, 1H), 8.33 (s, 1H), 4.43 (q, J=6 Hz, 2H), 4.36 (q, J=7 Hz, 2H), 1.63 (t, J=7 Hz, 3H), 1.43 (t, J=6 Hz, 3H).

### e) Preparation of Morpholino 6-chloro-1-ethyl-1,4-dihydro-4-oxo-1,7-naphthyridine-3-carboxamide

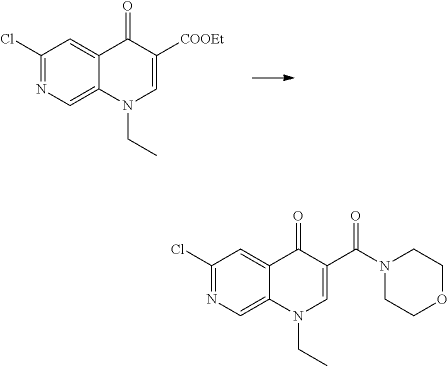 [SMILES: CCOC(=O)c1cn(CC)c2cnc(Cl)cc2c1=O ; CCn1cc(C(=O)N2CCOCC2)c(=O)c2cc(Cl)ncc21]

A mixture of trimethylaluminium (4 ml, 2M), morpholine (600 mg) in dry DCM (15 ml) were stirred for 1 h at 35° C. under nitrogen. After 1 h, ethyl 6-chloro-1-ethyl-1,4-dihydro-4-oxo-1,7-naphthyridine-3-carboxylate (900 mg) was added and the reaction mixture stirred o/n at the same temperature. Next day, 1M HCl (10 ml) was added carefully with stirring. After a standard ethyl acetate/aqueous work-up, the residue from the evaporated organic layer gave the morpholino 6-chloro-1-ethyl-1,4-dihydro-4-oxo-1,7-naphthyridine-3-carboxamide (700 mg).

ESIMS: m/z 322.0 [M+H]⁺.

¹H NMR (300 MHz, CDCl₃): δ 8.84 (s, 1H), 8.29 (s, 1H), 8.11 (s, 1H), 4.34 (q, J=7.3 Hz, 2H), 4.22 (m, 1H), 3.3-3.8 (m, 8H), 1.63 (t, J=7.3 Hz, 3H).

### f) Preparation of Morpholino 6-(2,3-Dihydro-1H-linden-2-ylamino)-1-ethyl-1,4-dihydro-4-oxo-1,7-naphthyridine-3-carboxamide (Example 3)

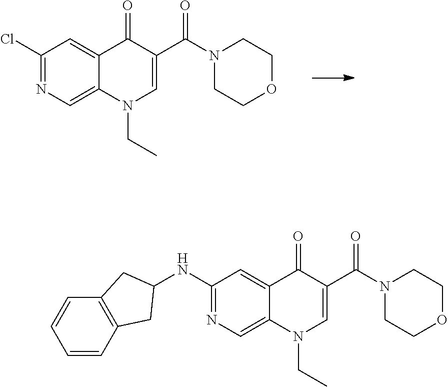 [SMILES: CCn1cc(C(=O)N2CCOCC2)c(=O)c2cc(Cl)ncc21 ; CCn1cc(C(=O)N2CCOCC2)c(=O)c2cc(NC3Cc4ccccc4C3)ncc21]

A mixture of 6-chloro-1-ethyl-1,4-dihydro-morpholino-4-oxo-1,7-naphthyridine-3-carboxamide (100 mg) and 2-aminoindane (in excess) was heated at 135° C. for 12 h. The reaction mixture was cooled to RT, ethyl acetate (100 ml) and water (20 ml) was added. The organic phase was separated, concentrated under reduced pressure and subjected to chromatography (SiO₂, 80% ethyl acetate in hexane) gave the product Example 3 (22% yield).

MS: m/z 419.0 [M+H]⁺.

¹H NMR (300 MHz, CDCl₃): δ 8.62 (s, 18, ArH), 8.01 (s, 1H, ArH), 7.28-7.18 (m, 5H, 5×ArH), 5.57 (bs, 1H, NH), 4.61 (m, 1H, NHCH), 4.27 (q, 2H, CH₂CH₃), 3.80 (m, 6H, morpholino), 3.49 (m, 4H, morpholino+CHCH₂), 2.98 (dd, 2H, CHCH₂), 1.60 (t, 3H, CH₃).

### Example 10

### Preparation of Diethylamino 6-(2,3-dihydro-1H-inden-2-ylamino)-1-ethyl-1,4-dihydro-4-oxo-1,8-naphthyridine-3-carboxamide (Example 10)

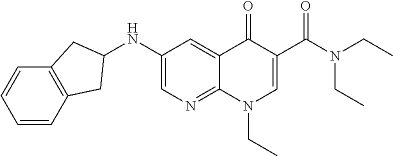 [SMILES: CCN(CC)C(=O)c1cn(CC)c2ncc(NC3Cc4ccccc4C3)cc2c1=O]

Trimethylaluminium (1 ml, 2M in toluene) was injected via syringe into a stirred solution of diethylamine (73 mg, 1 mmol) in DCM (10 ml). The reaction was stirred at room temperature for 1 h and then treated with ethyl 6-(2,3-dihydro-1H-inden-2-ylamino)-1,4-dihydro-4-oxo-1,5-naphthyridine-3-carboxylate (188.5 mg, 0.5 mmol). The resulting mixture was stirred at room temperature for 16 h and then poured into 5 ml of 2M HCl aq. solution. The organic compound was extracted with ethyl acetate (3×10 ml) and the combined extract was dried over MgSO₄, filtered and concentrated under reduced pressure to afford the crude oil (135 mg crude). Chromatography of the crude gave the desired product (100 mg)

ESIMS: m/z=405.0 [M+H]⁺.

¹H-NMR (300 MHz, CDCl₃): δ 8.15 (d, J=2.2 Hz, 1H), 7.85 (s, 18), 7.76 (d, J=2.2 Hz, 1H), 7.09-7.25 (m, 4H), 4.56 (d, J=5.6 Hz, 1H), 4.28-4.38 (m, 3H), 3.4-3.52 (m, 2H), 3.32-3.40 (m, 4H), 2.85 (dd, J=11, 3 Hz, 2H), 1.39 (t, J=5.6 Hz, 3H); 1.38 (t, J=5 Hz, 3H), 1.07 (t, J=5 Hz, 3H).

### Example 11

### Preparation of Ethyl 6-(3,4,5-trimethoxybenzoylamide)-1-ethyl-1,4-dihydro-4-oxo-1,8-naphthyridine-3-carboxylate (Example 11)

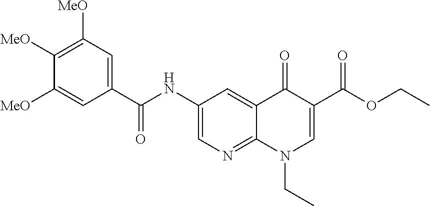 [SMILES: CCOC(=O)c1cn(CC)c2ncc(NC(=O)c3cc(OC)c(OC)c(OC)c3)cc2c1=O]

To the solution of ethyl 1-ethyl-1,4-dihydro-6-amino-4-oxo-1,8-naphthalidine-3-carboxylate (261 mg, 1 mmol) in dichloromethane (5 ml) was treated with 3,4,5-trimethoxybenzoyl chloride (460 mg, 2 mmol) at 5° C. The reaction mixture was then heated to 60° C. for 16 h. The reaction mixture was poured into ice and the product was extracted with ethyl acetate (3×10 ml). The combined extract was dried over MgSO₄ and concentrated under reduced pressure to afford the crude product (220 mg). Chromatography of a small quantity of the crude using 10% ethyl acetate in hexane afforded the desired product (30 mg).

ESIMS: m/z=455.9 [M+H]⁺.

¹H-NMR (300 MHz, CDCl₃): δ 9.69 (s, 1H), 9.42 (s, 1H), 8.87 (s, 1H), 8.68 (s, 1H), 7.24 (s, 1H), 7.14 (s, 1H), 4.51 (q, J=9, 5 Hz, 2H), 3.88 (s, 9H), 3.38-3.47 (m, 2H), 1.48 (t, J=6 Hz, 3H), 1.18 (t, J=6 Hz, 3H).

### Biological Data

### Effect of Compounds on Neurite Outgrowth

### 1. Cortical Neurons Culture

Female rats of 17 days gestation were killed by cervical dislocation and the foetuses were removed from the uterus. Their brains were placed in ice-cold medium of Leibovitz (L15, Gibco, Fisher bioblock, France). Cortex were dissected and meninges were carefully removed. The cortical neurons were dissociated by trypsinization for 30 min at 37° C. (trypsin-EDTA Gibco) in presence of 0.1 mg/ml DNAse I (Roche, France). The reaction was stopped by addition of in medium of Eagle modified by Dulbecco (DMEM; Gibco) with 10% of fetal bovine serum (FBS; Gibco). The suspension was triturated with a 10-ml pipette and using a needle syringe 21G and centrifuged at 350×g for 10 min at room temperature. The pellet of dissociated cells was resuspended in a medium consisting of Neurobasal (Gibco) supplemented with 2% B27 supplement (Gibco), 0.5 mM L-Glutamine (Gibco), an antibiotic-antimicotic mixture. Viable cells were counted in a Neubauer cytometer using the trypan blue exclusion test (Sigma). Cells were seeded on the basis of 30000 cells per Petri dish (ø35 mm, Nunc) precoated with poly-L-lysine.

### 2. Treatment

Cells were allowed to adhere 2 h and maintained in a humidified incubator at 37° C. in 5% CO₂-95% air atmosphere.

After neuronal adhesion (2 h after the plating), cultures were exposed to Compound 1 or BDNF for a period of 3 days.

### 3. Evaluation of Neurite Outgrowth

After the 3 days exposure of the neurons to the test compounds, cultures were washed with phosphate-buffered saline (PBS, Gibco) and fixed using 2.5% glutaraldehyde in PBS. Several pictures (˜80) of neurons with neurites without any branching were taken per condition using a digital camera (Coolpix 995; Nikon) mounted on the microscope (Nikon, objective 40×). Neurites were outlined on computer screen using imaging software (Image-Pro Plus, France), which automatically calculates the length.

### 4. Statistical Analysis

A global analysis of the data was performed using a one way analysis of variance (ANOVA), followed by Fisher's Protected Least Significant Difference when applicable. The level of significance was set to p<0.05. All results were expressed as mean±sem. N=˜180 cells

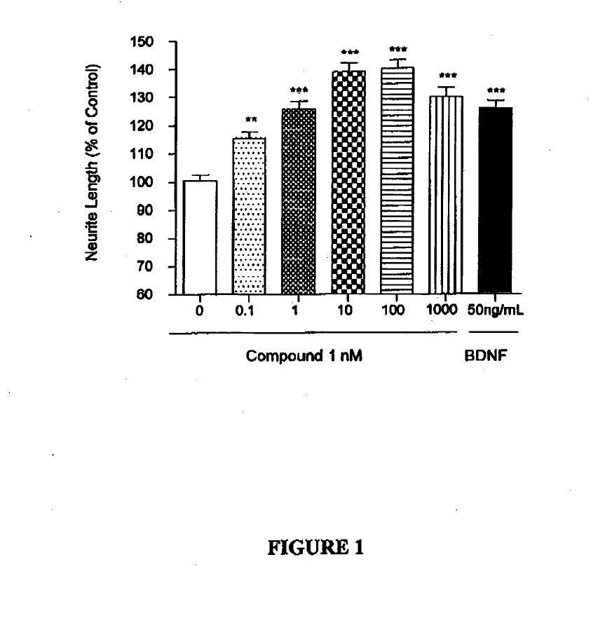

The compound of example 1h was tested at 0.1 nM, 1 nM, 10 nM, 100 nM and 1000 nM on two independent cultures comprising 2 Petri dishes per culture and per condition. In parallel, BDNF was tested at 50 ng/ml. The results are shown in FIG. 1.

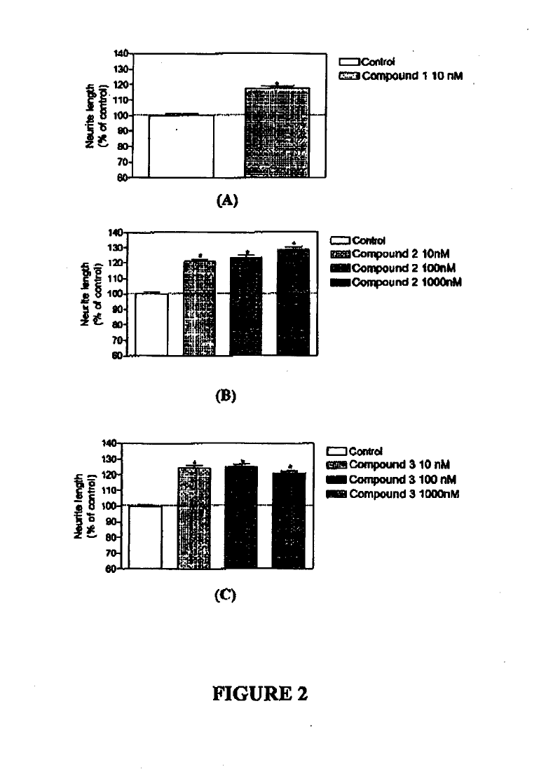

Results for experiments conducted on Examples 2 and 3 (relative to control and Example 1) are shown in FIGS. 2(A), 2(B), and 2(C).

The compounds of Examples 1h, 2, 3, 4, 5, 6, 7, 1f, 1g were evaluated in the neurite outgrowth assay and each compound gave significant neurite outgrowth at concentrations less than 100 nM.

### Investigation of Pathways Involved in Example 1's Effect on Neurite Outgrowth

The effect of Compound 1 on pathways involved in the production of neurites from rat primary cortical neurons was investigated using pathway specific inhibitors (Table 1).

TABLE 1
Compounds used to inhibit specific pathways in the Trk signalling

| Inhibitor | Target |
|---|---|
| U73122 | Phospholipase C |
| K252a | Trk receptor |
| Wortmannin | Phosphoinositide 3 kinase |
| PD98059 | MAPK |

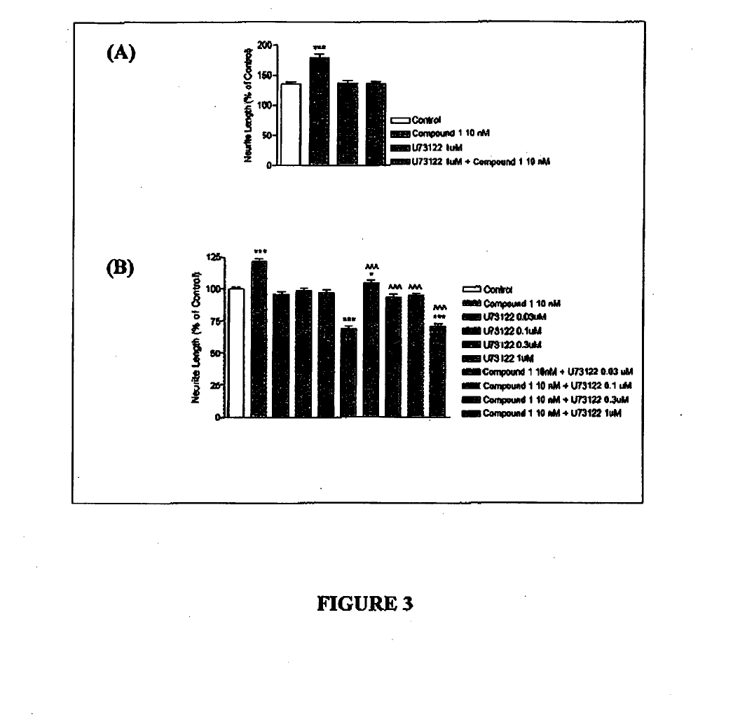

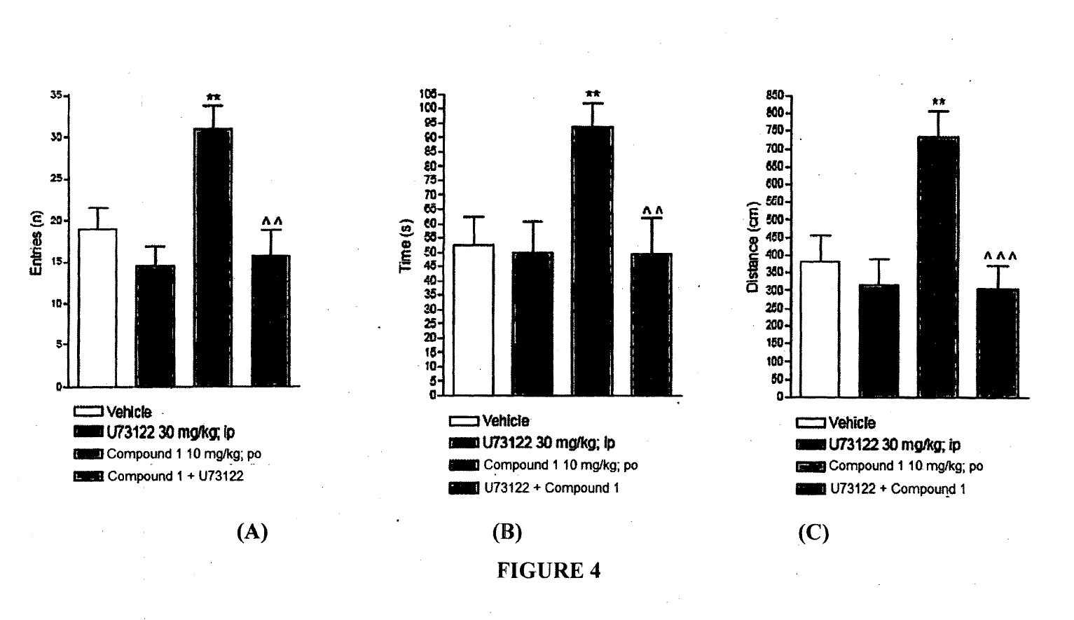

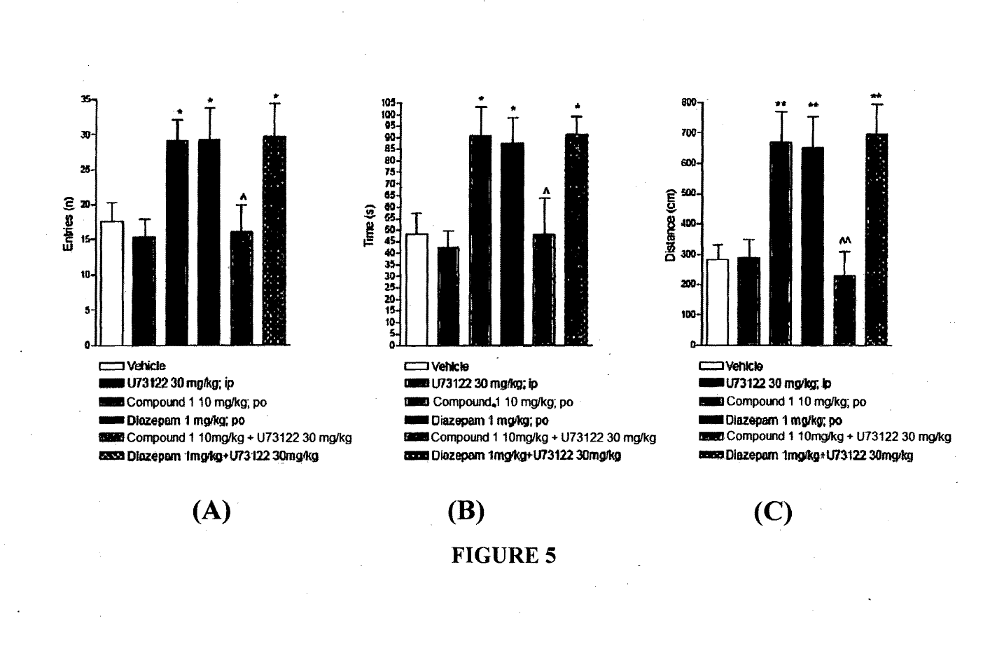

The phospholipase C inhibitor, U73122, produced significant block of Compound 1 activity at 1 μM. When tested over the range of 30 nM to 1 μM, a dose-related block was seen with full block occurring at 100 nM (FIGS. 3a and 3b). U73122 was then examined for an in vivo effect on the activity of Example 1 in the mouse light dark box. U73122 was given IP at 30 mg/kg, 120 minutes prior to testing. Compound 1 was dosed orally at 10 mg/kg, 60 minutes prior to testing. Compound 1 demonstrated robust anxiolytic activity. U73122 did not have any effect on the Time, Entries or Distance parameters but, when used in combination with 10 mg/kg of Compound 1, it fully blocked the anxiolytic effect (FIG. 4). This experiment was repeated using the anxiolytic compound diazepam to determine whether the activity of U73122 was specific to Compound 1 (FIG. 5). U73122 did not cause any change to the animals' responses to diazepam but once again fully blocked the Compound 1 responses (FIG. 4).

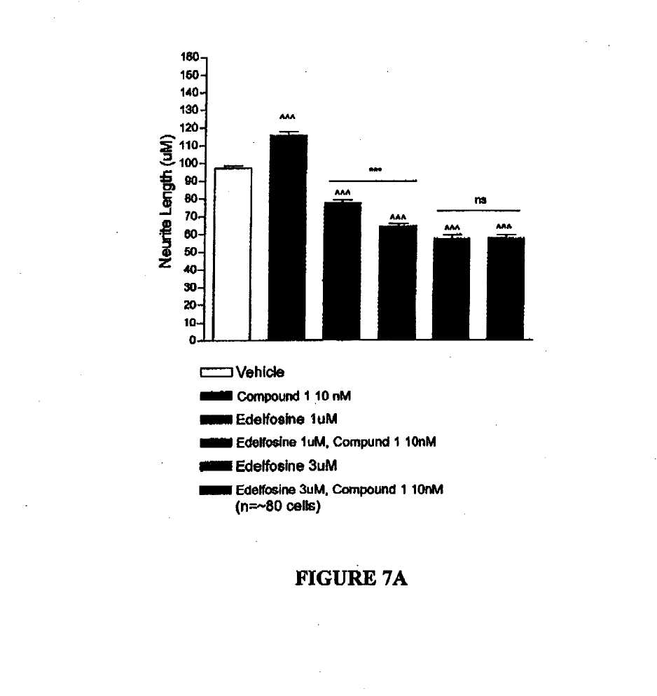

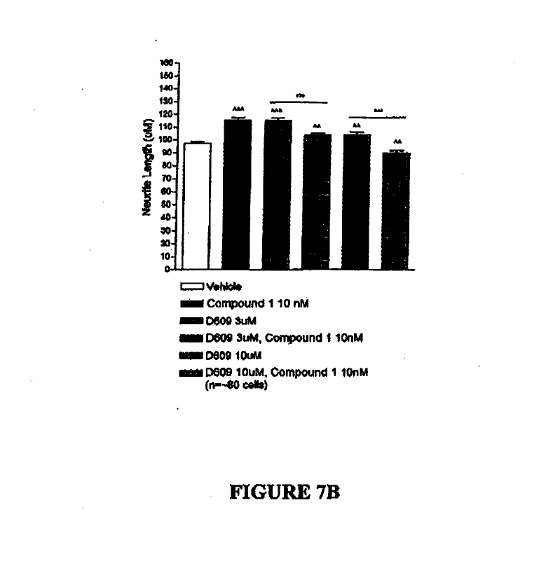

This result was confirmed using two other inhibitors of phospholipase C enzymes, D609, and Edelfosine, which also blocked Compound 1's activity in the neurite outgrowth assay. While doses of Edelfosine reduced neurite outgrowth and doses of D609 enhanced neurite outgrowth, when either compound was used in combination with Compound 1, a significant and dose-dependant reduction of Compound 1's effect was seen (FIGS. 7A and 7B).

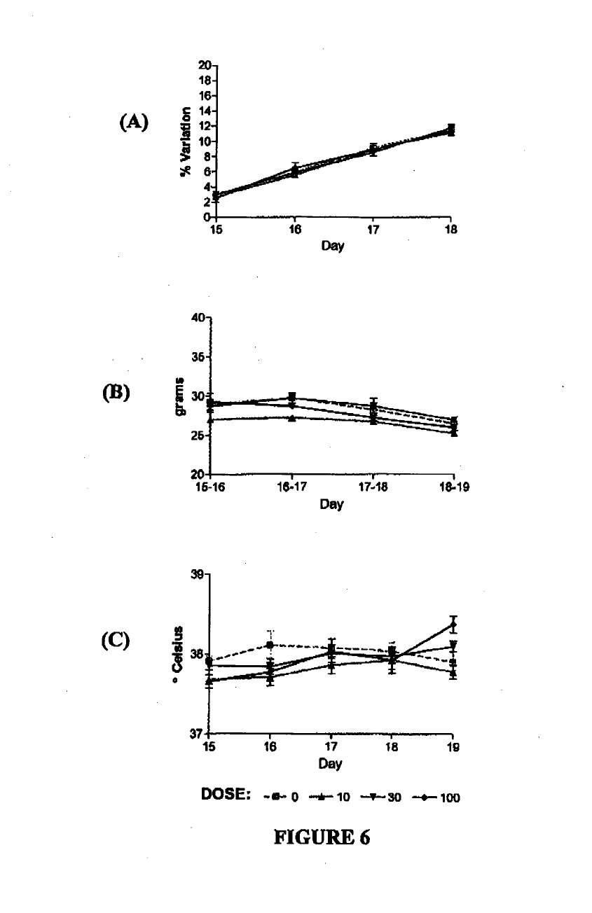

Rats treated chronically (14-20 days) with opioids, benzodiazepines Or SSRIs display adverse physical effects after non-precipitated withdrawal of the drugs. The potential consequences of abrupt cessation of dosing with Compound 1 was assessed following 14 days of treatment at 0, 10, 30 and 100 mg/kg/day. Withdrawal of Compound 1 treatment did not produce significant changes in body temperature, weight gain or food consumption compared to the no-drug treatment group during the post-treatment period (5 days) (FIG. 6). These findings indicate that repeat dosing with Compound 1 does not cause the development of physical dependence on the drug and is consistent with its suitability for chronic use.

1. A method of enhancing neurite outgrowth in a subject in need thereof, the method comprising administering to said subject an effective amount of a compound of formula (I) or pharmaceutically acceptable salt thereof: 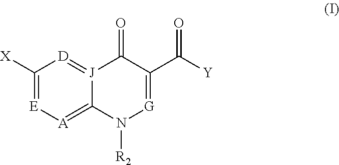
where
A, E, G and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N;
J represents C or N;
X represents halogen, heteroaromatic, OR₁, or NR₁R″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heterocyclyl, optionally substituted heteroaryl, optionally substituted oxysulfinyl, optionally substituted oxysulfonyl, optionally substituted sulfinyl, and optionally substituted sulfonyl);
Y represents OR′″ (where R′″ is H or optionally substituted alkyl) or NR₃R₄;
R₁ represents H, optionally substituted cycloalkyl, optionally substituted cycloalkenyl, optionally substituted alkyl, optionally substituted acyl, optionally substituted aryl, optionally substituted heterocyclyl, or optionally substituted heteroaryl;
R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
R₃ and R₄ each independently represent H, optionally substituted alkyl, optionally substituted cycloalkyl, optionally substituted aryl, optionally substituted heteroaryl, optionally substituted heterocyclyl, or together with the N-atom represent an optionally substituted N-containing heteroaryl or optionally substituted N-containing heterocyclyl.

2. The method of claim 1, wherein the subject has a neurodegenerative disease.

3. The method of claim 2, wherein the neurodegenerative disease is selected from the group consisting of AIDS dementia complex, adrenoleukodystrophy, Alexander disease, Alpers' disease, amyotrophic lateral sclerosis, ataxia telangiectasia, Batten disease, bovine spongiform encephalopathy, Canavan disease, corticobasal degeneration, Creutzfeldt-Jakob disease, dementia with Lewy bodies, fatal familial insomnia, frontotemporal lobar degeneration, Huntington's disease, infantile Refsum disease, Kennedy's disease, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progranulin, progressive supranuclear palsy, protein aggregation, Refsum disease, Sandhoff disease, diffuse myelinoclastic sclerosis, Shy-Drager syndrome, spinocerebellar ataxia, subacute combined degeneration of spinal cord, Tabes dorsalis, Tay-Sachs disease, toxic encephalopathy, transmissible spongiform encephalopathy, and Wobbly hedgehog syndrome.

4. A method for the treatment or prophylaxis of depression in a subject in need thereof, or treating or preventing relapse of depression in a subject receiving antidepressant therapy, the method comprising administering to said subject a compound of formula (I) or a pharmaceutically acceptable salt thereof; 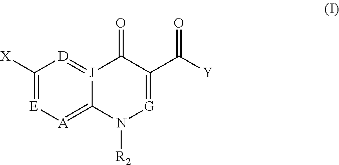
where
A, E, G and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N;
J represents C or N;
X represents halogen, heteroaromatic, OR₁, or NR₁R″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heterocyclyl, optionally substituted heteroaryl, optionally substituted oxysulfinyl, optionally substituted oxysulfonyl, optionally substituted sulfinyl, and optionally substituted sulfonyl);
Y represents OR′″ (where R′″ is H or optionally substituted alkyl) or NR₃R₄;
R₁ represents H, optionally substituted cycloalkyl, optionally substituted cycloalkenyl, optionally substituted alkyl, optionally substituted acyl, optionally substituted aryl, optionally substituted heterocyclyl, or optionally substituted heteroaryl;
R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
R₃ and R₄ each independently represent H, optionally substituted alkyl, optionally substituted cycloalkyl, optionally substituted aryl, optionally substituted heteroaryl, optionally substituted heterocyclyl, or together with the N-atom represent an optionally substituted N-containing heteroaryl or optionally substituted N-containing heterocyclyl.

5-12. (canceled)

13. A method of treating or preventing a neurodegenerative disease, the method comprising administering to said subject a compound of formula (I) or a pharmaceutically acceptable salt thereof; 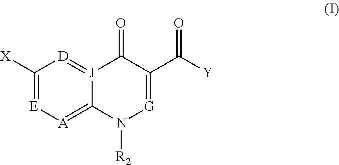
where
A, E, G and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N;
J represents C or N;
X represents halogen, heteroaromatic, OR₁, or NR₁R″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heterocyclyl, optionally substituted heteroaryl, optionally substituted oxysulfinyl, optionally substituted oxysulfonyl, optionally substituted sulfinyl, and optionally substituted sulfonyl);
Y represents OR′″ (where R′″ is H or optionally substituted alkyl) or NR₃R₄;
R₁ represents H, optionally substituted cycloalkyl, optionally substituted cycloalkenyl, optionally substituted alkyl, optionally substituted acyl, optionally substituted aryl, optionally substituted heterocyclyl, or optionally substituted heteroaryl;
R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
R₃ and R₄ each independently represent H, optionally substituted alkyl, optionally substituted cycloalkyl, optionally substituted aryl, optionally substituted heteroaryl, optionally substituted heterocyclyl, or together with the N-atom represent an optionally substituted N-containing heteroaryl or optionally substituted N-containing heterocyclyl.

14. The method of claim 13, wherein the neurodegenerative disease is selected from the group consisting of AIDS dementia complex, adrenoleukodystrophy, Alexander disease, Alpers' disease, amyotrophic lateral sclerosis, ataxia telangiectasia, Batten disease, bovine spongiform encephalopathy, Canavan disease, corticobasal degeneration, Creutzfeldt-Jakob disease, dementia with Lewy bodies, fatal familial insomnia, frontotemporal lobar degeneration, Huntington's disease, infantile Refsum disease, Kennedy's disease, Krabbe disease, Lyme disease, Machado-Joseph disease, multiple sclerosis, multiple system atrophy, neuroacanthocytosis, Niemann-Pick disease, Parkinson's disease, Pick's disease, primary lateral sclerosis, progranulin, progressive supranuclear palsy, protein aggregation, Refsum disease, Sandhoff disease, diffuse myelinoclastic sclerosis, Shy-Drager syndrome, spinocerebellar ataxia, subacute combined degeneration of spinal cord, Tabes dorsalis, Tay-Sachs disease, toxic encephalopathy, transmissible spongiform encephalopathy, and Wobbly hedgehog syndrome.

15. (canceled)

16. The method of claim 1, wherein the compound is of formula (Ia), (Ib), (Ic), (Id), (Ie), or (If): 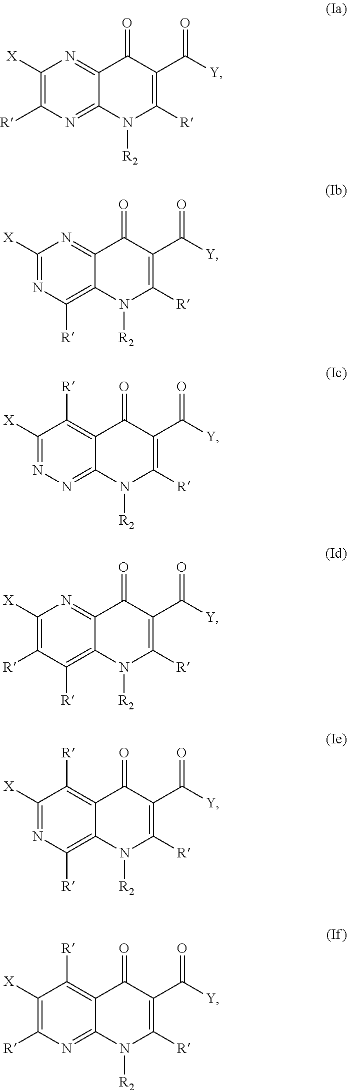
or a pharmaceutically acceptable salt thereof.

17. (canceled)

18. The method of claim 1, wherein R′ is selected from the group consisting of hydrogen; halogen; cyano; nitro; alkyl; substituted alkyl; optionally substituted aryl; optionally substituted acyl; optionally substituted alkoxy; oxyacyl; acyloxy; optionally substituted arylalkyl; optionally substituted sulfinyl; optionally substituted sulfonyl; optionally substituted oxyacylamino; optionally substituted oxythioacyl; optionally substituted thioacyloxy; optionally substituted sulphinylamino; amino; substituted amino; optionally substituted sulphonylamino; optionally substituted thio; optionally substituted oxysulfinylamino; optionally substituted oxysulfonylamino; optionally substituted alkenyl; and optionally substituted alkynyl.

19. (canceled)

20. The method of claim 1, wherein Y is NR₃R₄.

21. The method of claim 20, wherein one of R₃ and R₄ is H and the other is optionally substituted alkyl, optionally substituted aryl, optionally substituted C₃₋₇ cycloalkyl, optionally substituted heteroaryl, or optionally substituted heterocyclyl.

22. The method of claim 21, wherein both R₃ and R₄ are each independently optionally substituted C₁₋₃ alkyl.

23. (canceled)

24. The method of claim 1, wherein the compound is of formula (I′) or a salt thereof: 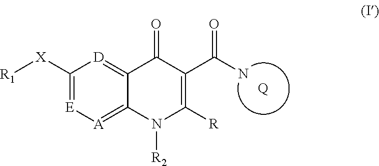
where
A, E, and D are independently CR′ (where R′ is selected from H, carboxyl, cyano, dihalomethoxy, halogen, hydroxy, nitro, pentahaloethyl, phosphono, phosphorylamino, phosphinyl, sulfo, trihaloethenyl, trihalomethanethio, trihalomethyl, trihalomethoxy, optionally substituted acyl, optionally substituted acylamino, optionally substituted acylimino, optionally substituted acyliminoxy, optionally substituted acyloxy, optionally substituted arylalkyl, optionally substituted arylalkoxy, optionally substituted alkenyl, optionally substituted alkenyloxy, optionally substituted alkoxy, optionally substituted alkyl, optionally substituted alkynyl, optionally substituted alkynyloxy, optionally substituted amino, optionally substituted aminoacyl, optionally substituted aminoacyloxy, optionally substituted aminosulfonyl, optionally substituted aminothioacyl, optionally substituted aryl, optionally substituted arylamino, optionally substituted aryloxy, optionally substituted cycloalkenyl, optionally substituted cycloalkyl, optionally substituted heteroaryl, optionally substituted heterocyclyl, optionally substituted oxyacyl, optionally substituted oxyacylamino, optionally substituted oxyacyloxy, optionally substituted oxyacylimino, optionally substituted oxysulfinylamino, optionally substituted oxysulfonylamino, optionally substituted oxythioacyl, optionally substituted oxythioacyloxy, optionally substituted sulfinyl, optionally substituted sulfinylamino, optionally substituted sulfonyl, optionally substituted sulphonylamino, optionally substituted thio, optionally substituted thioacyl, and optionally substituted thioacylamino) or N, and wherein at least one of A, E and D is N;
X represents O or NR″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heterocyclyl, optionally substituted heteroaryl, optionally substituted oxysulfinyl, optionally substituted oxysulfonyl, optionally substituted sulfinyl, and optionally substituted sulfonyl);
R represents H or optionally substituted alkyl;
R₁ represents optionally substituted cycloalkyl, optionally substituted alkyl, optionally substituted acyl, optionally substituted aryl, optionally substituted heterocyclyl, or optionally substituted heteroaryl;
R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
Q represents an optionally substituted N-containing heterocyclyl or an optionally substituted N-containing heteroaryl.

25. The method of claim 24, wherein the compound is of formula (I′a), (I′b), (I′c), (I′d), (I′e), or (I′f): 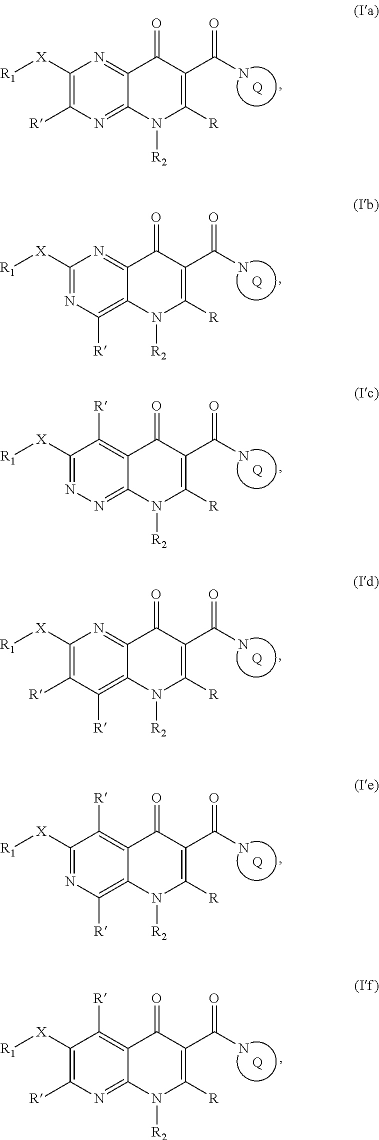
or a pharmaceutically acceptable salt thereof.

26-27. (canceled)

28. The method of claim 24, wherein Q is selected from the group consisting of substituted N-containing heterocyclyl, N-containing heterocyclyl, and morpholinyl.

29. The method of claim 1, wherein X is NR″ where R″ is hydrogen, C₁₋₃ alkyl, benzyl, or acetyl.

30-33. (canceled)

34. The method of claim 1, wherein R₁ is optionally substituted alkyl, optionally substituted cycloalkyl, or optionally substituted cycloalkenyl, wherein if substituted a substituent is selected from the group consisting of optionally substituted acyl, optionally substituted aryl, halogen, COOH, NH₂, methoxy, mono or dialkyl amino, and CF₃.

35. The method of claim 1, wherein R₁ is benzofused C₅-C₇ cycloalkyl.

36. The method of claim 35, wherein R₁ is indanyl or 1,2,3,4-tetrahydronaphthalenyl.

37. The method of claim 35, wherein R₁ is

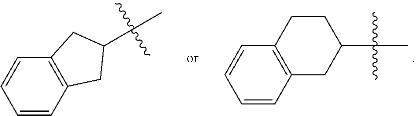 [SMILES: *C1Cc2ccccc2C1 ; *C1CCc2ccccc2C1]

38. (canceled)

39. The method of claim 1, wherein R₂ is hydrogen, C₁₋₆ alkyl, benzyl or acetyl.

40. (canceled)

41. The method of claim 1, wherein the compound of formula (I) is a compound of formula (I″f) 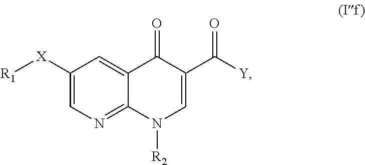
wherein:
X represents O or NR″ (where R″ is selected from H, optionally substituted alkyl, optionally substituted aryl, optionally substituted cycloalkyl, optionally substituted acyl, optionally substituted alkenyl, optionally substituted heterocyclyl, optionally substituted heterocyclyl, optionally substituted heteroaryl, optionally substituted oxysulfinyl, optionally substituted oxysulfonyl, optionally substituted sulfinyl, and optionally substituted sulfonyl);
Y represents OR′″ (where R′″ is H or optionally substituted alkyl) or NR₃R₄;
R₁ represents H, optionally substituted cycloalkyl, optionally substituted cycloalkenyl, optionally substituted alkyl, optionally substituted acyl, optionally substituted aryl, optionally substituted heterocyclyl, or optionally substituted heteroaryl;
R₂ represents H, optionally substituted cycloalkyl, optionally substituted alkyl, optionally substituted acyl, optionally substituted aryl, optionally substituted alkenyl, optionally substituted heterocyclyl, optionally substituted heteroaryl, optionally substituted oxysulfinyl, optionally substituted oxysulfonyl, optionally substituted sulfinyl, or optionally substituted sulfonyl; and
R₃ and R₄ each independently represent H, optionally substituted alkyl, optionally substituted cycloalkyl, optionally substituted aryl, optionally substituted heteroaryl, optionally substituted heterocyclyl, or together with the N-atom represents an optionally substituted N-containing heteroaryl or optionally substituted N-containing heterocyclyl.

42. The method of claim 41, wherein:
Y is —OC₁-C₆ alkyl or NR₃R₄;
R₃ is independently H or C₁-C₆ alkyl and R₄ is C₁-C₆ alkyl, or R₃ and R₄ together with the N-atom form an optionally substituted N-containing heteroaryl or optionally substituted N-containing heterocyclyl;
R₁X is —NH₂, —NH(C₁-C₆ alkyl), —N(C₁-C₆ alkyl)₂, —NH(benzofused C₅-C₇ cycloalkyl), —NHCO(C₁-C₆ alkyl) or —NHCO (optionally substituted aryl); and
R₂ is C₁-C₃ alkyl.

43. (canceled)

44. The method of claim 42, wherein R₁X is

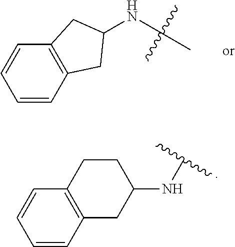 [SMILES: *NC1Cc2ccccc2C1 ; *NC1CCc2ccccc2C1]

45. (canceled)

46. The method of claim 1, wherein the compound is selected from the group consisting of: 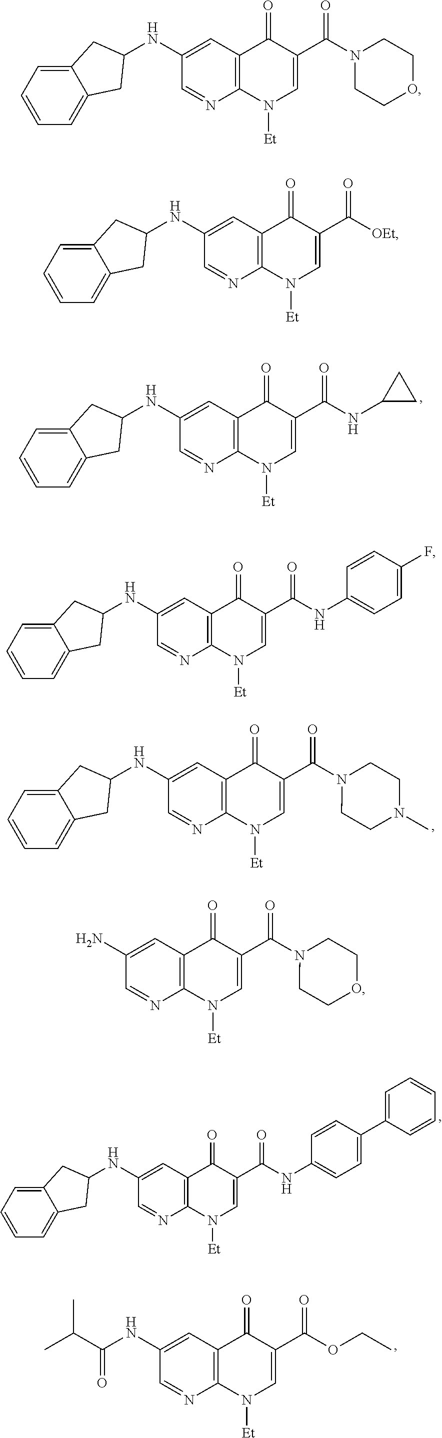 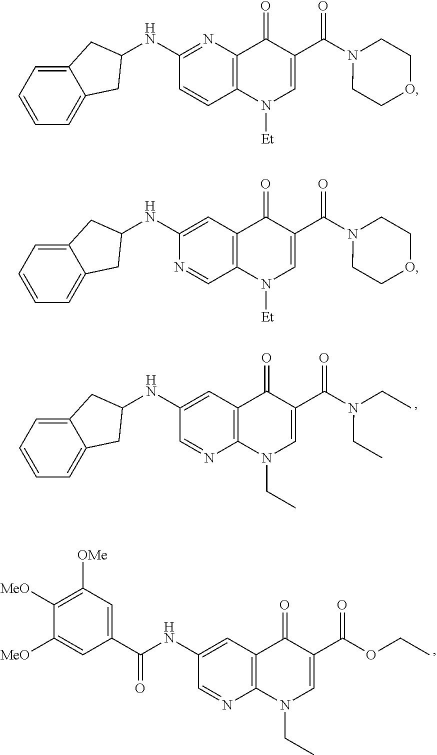
and pharmaceutically acceptable salts thereof.

47. (canceled)
